(19) United States
(12) Patent Application Publication
(10) Pub. No.: US 2025/0346582 A1
(43) Pub. Date: Nov. 13, 2025

(54) SPLEEN TYROSINE KINASE INHIBITORS AND METHODS OF USE THEREOF

(71) Applicant: THE BOARD OF TRUSTEES OF THE UNIVERSITY OF ILLINOIS, URBANA, IL (US)

(72) Inventors: WONHWA CHO, CHICAGO, IL (US); ASHUTOSH SHARMA, CHICAGO, IL (US)

(21) Appl. No.: 18/860,031

(22) PCT Filed: May 24, 2023
(86) PCT No.: PCT/US2023/067391

§ 371 Date: Oct. 25, 2024

Related U.S. Application Data
(60) Provisional application No. 63/345,072, filed on May 24, 2022.

(51) Int. Cl. C07D 405/12; A61K 31/4402; A61K 31/497; A61K 31/506; A61K 31/675; A61P 35/02; C07D 213/74; C07D 239/42; C07D 241/20; C07D 403/12; C07D 409/12; C07F 9/58

(52) U.S. Cl. CPC C07D 405/12; A61K 31/4402; A61K 31/497; A61K 31/506; A61K 31/675; A61P 35/02; C07D 213/74; C07D 239/42; C07D 241/20; C07D 403/12; C07D 409/12; C07F 9/58

(57) ABSTRACT

In accordance with the purpose(s) of the present disclosure, as embodied and broadly described herein, the disclosure, in one aspect, relates to compounds (I) that are inhibitors for spleen tyrosine kinase (Syk), which is a key signaling protein in hematologic cells and implicated in multiple hematopoietic malignancies, cancer (e.g., chronic lymphoid leukemia (CLL) or acute myeloid leukemia (AML)), diabetes, and immune disorders. In one aspect, the compounds described herein drug resistance, which renders current ATP-competitive Syk inhibitors ineffective.

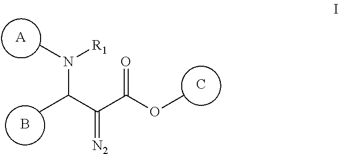

### CROSS-REFERENCE TO RELATED APPLICATIONS

This application claims the benefit of and priority to co-pending U.S. Provisional Patent Application No. 63/345,072, filed on May 24, 2022, the contents of which are incorporated by reference herein in their entireties.

### STATEMENT REGARDING FEDERALLY SPONSORED RESEARCH OR DEVELOPMENT

This invention was made with government support under grant number R35 GM122530 awarded by the National Institutes of Health. The government has certain rights in this invention.

### BACKGROUND

Recent advances in structural and chemical biology have made it possible to develop new small molecule drugs against pharmacological targets traditionally considered undruggable¹⁻³. However, there still are many targets that are not easily accessible by current approaches. Also, drug resistance remains a major challenge for all targeted therapies⁴⁻⁶. Therefore, search continues for new and alternative methods for drug development.

Lipid-protein interaction plays key regulatory roles in diverse biological processes, including cell signaling^{7,8}, and dysregulated lipid-protein interaction has been linked to numerous human diseases^{9,10}. Thus, the lipid binding sites of cell signaling proteins, including kinases, are attractive targets for novel drug development¹¹⁻¹³. Nevertheless, lipid-protein interaction has not been successfully targeted for drug discovery for various conceptual and technical reasons. An initial interest in developing lipid-protein interaction inhibitors was dampened by two notions. First, it was thought that only a limited number of proteins, such as pleckstrin homology (PH) domain-containing Akt, specifically interact with membrane lipids^{7,14}. It was also thought that these proteins must be modulated by lipid-like inhibitors that are often difficult to synthesize and have undesirable properties, such as low water solubility¹³.

Development of lipid-protein interaction inhibitors has been further hampered by multiple technical factors. They include the difficulties in understanding the detailed mechanisms of lipid-protein interaction in the natural membrane environment by conventional structural biology techniques^{7,15}, the absence of proper small molecule libraries covering the yet-undefined chemical space of lipid-protein interfaces, and a dearth of robust and versatile high-throughput lipid-protein binding assays⁸.

### SUMMARY

In accordance with the purpose(s) of the present disclosure, as embodied and broadly described herein, the disclosure, in one aspect, relates to compounds that are inhibitors for spleen tyrosine kinase (Syk), which is a key signaling protein in hematologic cells and implicated in multiple hematopoietic malignancies, cancer (e.g., chronic lymphoid leukemia (CLL) or acute myeloid leukemia (AML)), diabetes, and immune disorders. In one aspect, the compounds described herein drug resistance, which renders current ATP-competitive Syk inhibitors ineffective.

In one aspect, the compounds have the formula I, or a pharmaceutically acceptable salt thereof

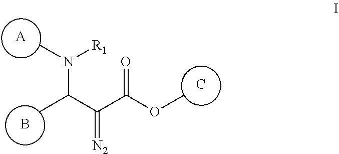

wherein

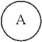

is 5 or 6 membered substituted or unsubstituted heteroaryl;

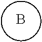

is 5 to 10 membered substituted or unsubstituted heteroaryl or substituted or unsubstituted phenyl;

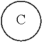

is cycloalkyl; and
R₁ is H or C₁-C₄ alkyl.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1C show the efficacy and specificity of Syk inhibitors. (a) Cellular efficacy of WC35, WC36, and WC38 was determined by dose-dependent inhibition of Syk phosphorylation in Raji B cells. (b) Iₘₐₓ and IC₅₀ values were determined by non-linear least-squares analysis of data using the equation: I=Iₘₐₓ/(1+IC₅₀/[inhibitor]) where I indicates % inhibition. % inhibition was calculated by 100×(relative decrease in pSyk/(total Syk) at a given inhibitor concentration ([inhibitor]) compared to that without an inhibitor. Each data represents average ±s.d. from triplicate measurements. (c) Specificity of WC35, WC36, WC38, and entospletinib (ENTO) was determined by measuring the inhibition of ZAP70 phosphorylation by these molecules (5 μM overnight) in Jurkat T cells. Jurkat T cells were stimulated by OKT3.

FIGS. 2A-2D show the inhibition of the signaling activity and proliferation of MV4-11 AML cells by Syk-cSH2 inhibitors. (a) Western blot analysis of IgG2-stimulated (10 ng/ml for 10 min) phosphorylation of Syk, STAT3/STAT5 and ERK1/2 after MV4-11 cells were pre-treated with 5 μM of WC35, WC36, WC38, and entospletinib (ENTO) overnight. GAPDH was used as a gel loading control. (b) Dose dependent inhibition of the proliferation of MV4-11 cells by WC35, WC36, WC38, and entospletinib measured by the XTT assay. Imax and IC50 values were determined by non-linear least-squares analysis of data using the equation: I=Imax/(1+IC50/[inhibitor]) where I indicates % inhibition at a given inhibitor concentration ([inhibitor]). (c) Inhibition of IgG2-stimulated phosphorylation of Syk, STAT3/STAT5 and ERK1/2 in entospletinib-resistant MV-4-11 cells by 5 μM of WC35, WC36, and WC38. (d) Dose dependent inhibition of the proliferation of entospletinib-resistant MV4-11 cells by WC36 and entospletinib.

FIGS. 3A-3D show the inhibition of the signaling activity and proliferation of HL-60 AML cells by Syk-cSH2 inhibitors. (a) Inhibition of IgG2-stimulated (10 ng/ml for 10 min) phosphorylation of Syk, STAT3, STAT5 and ERK1/2 in HL-60 cells by entospletinib (ENTO), WC36, WC35, and WC38 (5 μM each). DMSO was used as a negative control and GAPDH as a gel loading control. (b) Dose dependent inhibition of the proliferation of HL-60 cells by WC36 and entospletinib measured by the XTT assay. (c) Inhibition of phosphorylation of Syk, STAT3/5 and ERK1/2 in patient-derived primary AML cells by WC36. Cells were cultured overnight in the presence of CD34+ cytokine supplement. DMSO was used as a negative control. Representative data from one (Patient 1) of four patient samples are shown here. (d) Inhibition of the proliferation of four patient AML cells by WC36 measured by the colony growth assay. The number of colonies after WC36 treatment was normalized to the enumerated colonies after DMSO treatment.

FIG. 4 shows the scaffolding function of Syk and its inhibition by WC36. Immunoprecipitation (IP) with the anti-Syk antibody showed that Syk interacted with STAT3/5 and ERK1/2, which was potently inhibited by 5 μM WC36, but not by 5 μM entospletinib in both entospletinib (ENT)-resistant and naive MV4-11 cells. The expression level of RAS was much lower in naive MV4-11 cells than in entospletinib-resistant cells. Also, RAS was included in the Syk complex only in entospletinib-resistant cells.

FIGS. 5A-5B show cellular target validation of WC36. (a) Inhibition of Syk WT and WC36-binding site mutants by WC36. Entospletinib-resistant MV4-11 cells were treated with shRNA to suppress the expression of endogenous Syk and then transfected with exogenous Syk-WT and four Syk-E614X genes, respectively. These constructs harbor silent mutations to avoid gene suppression by Syk ShRNA. Phosphorylation of Syk and ERK1/2 in these cells was then monitored before and after treatment with 5 μM WC36 overnight. The expression level of each protein in all analyzed cells was comparable as shown in total protein blotting data. (b) Western blot analysis of WC36B-binding proteins captured by pull-down by streptavidin beads. Biotin was used as a negative control. GAPDH was used as a gel loading control.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The position of a substituent can be defined relative to the positions of other substituents in an aromatic ring. For example, as shown below in relationship to the “R” group, a second substituent can be “ortho,” “para,” or “meta” to the R group, meaning that the second substituent is bonded to a carbon labeled ortho, para, or meta as indicated below. Combinations of ortho, para, and meta substituents relative to a given group or substituent are also envisioned and should be considered to be disclosed.

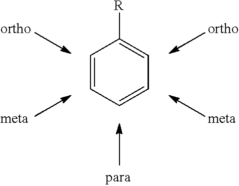

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cyclooctynyl, cyclononynyl, and the like. The term “heterocycloalkynyl” is a type of cycloalkenyl group as defined above and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A'S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

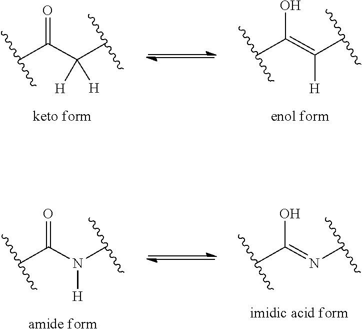 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

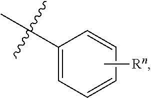

which is understood to be equivalent to a formula:

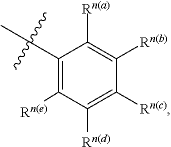

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a hematological malignancy, breast cancer, and/or another solid malignancy. The effect can be therapeutic in terms of a partial or complete cure of a disease, condition, symptom or adverse effect attributed to the disease, disorder, or condition. The term “treatment” as used herein can include any treatment of a hematological malignancy, breast cancer, and/or another solid tumor in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Compounds and Methods of Making and Using the Compounds

In one aspect, disclosed herein is a compound having a structure according to structure I or the pharmaceutically acceptable salt thereof

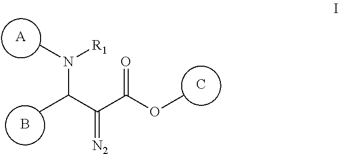

wherein

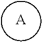

is 5 or 6 membered substituted or unsubstituted heteroaryl;

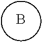

is 5 to 10 membered substituted or unsubstituted heteroaryl or substituted or unsubstituted phenyl; and

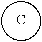

is cycloalkyl;
R₁ is H or C₁-C₄ alkyl.

In one aspect,

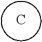

is cyclohexyl in structure I. In another aspect,

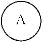

is a 6-membered substituted or unsubstituted heteroaryl in structure I. In another aspect,

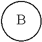

is a 5 or 9-membered substituted or unsubstituted heteroaryl in structure I. In another aspect,

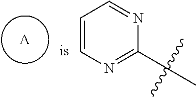

in structure I.

In another aspect,

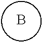

in structure I is

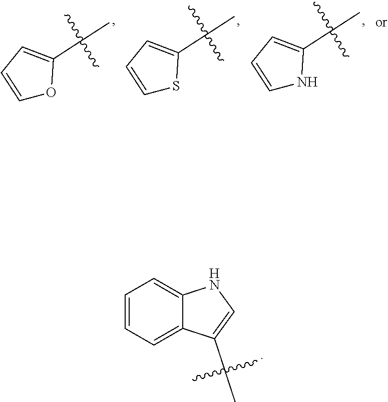 [SMILES: *c1ccco1 ; *c1cccs1 ; *c1ccc[nH]1 ; *c1c[nH]c2ccccc12]

In another aspect,

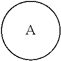

is a 6-membered substituted or unsubstituted heteroaryl,

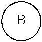

is a 5 membered substituted or unsubstituted heteroaryl or a 9 membered fused heteroaryl,

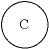

is cyclohexyl, and
R¹ is hydrogen.

In another aspect, the compound has the structure II or the pharmaceutically acceptable salt thereof

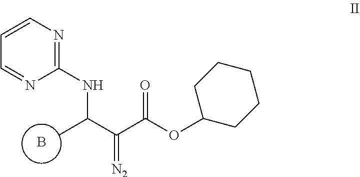

In one aspect,

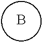

is a 5 membered substituted or unsubstituted heteroaryl in structure II. In another aspect,

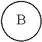

is a 5 membered substituted or unsubstituted heteroaryl in structure II, wherein the 5 membered heteroaryl is a substituted or unsubstituted furan, a substituted or unsubstituted pyrrole, or a substituted or unsubstituted thiophene or an unsubstituted furan or an unsubstituted pyrrole.

In another aspect,

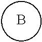

is a 9 membered fused substituted or unsubstituted heteroaryl in structure II. In another aspect,

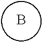

is a 9 membered fused substituted or unsubstituted heteroaryl in structure II, wherein the 9 membered substituted or unsubstituted fused heteroaryl is a substituted or unsubstituted indole, a substituted or unsubstituted isoindole, a substituted or unsubstituted indolizine, a substituted or unsubstituted purine, or a substituted or unsubstituted indole.

In another aspect, the compound has the following structure

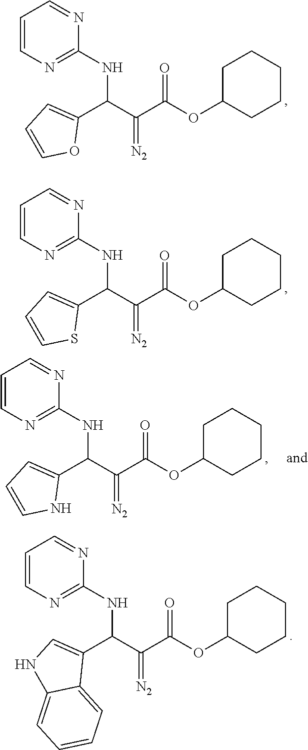

### General Synthetic Method

In one aspect, the compounds described herein can be produced by reacting an aldehyde III with an amino compound IV and a diazoacetate compound V as depicted in the reaction scheme below.

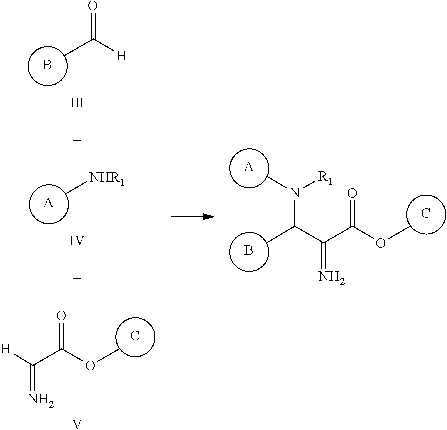

wherein the variables in structures III, IV, and V are as defined above.

Exemplary methods for producing compounds described herein, as well as characterization information, are provided in the Examples. Solvents, temperatures, presence or absence of protecting groups, and other reaction conditions may vary according to the specific substituents in the compound being synthesized.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentaerythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethyl carbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and dibutylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentaacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In one aspect, an appropriate dosage level will generally be about 0.01 to 1000 mg of a compound described herein per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Methods of Use

A large number of SH2 domain-containing kinases have been implicated in human diseases, including cancer, diabetes, and immune disorders and thus have been targets for active drug development²⁴. To date most small molecule kinase inhibitors have been designed to target the ATP-binding pockets in their kinase domains^{25,26}. Some of these ATP-competitive inhibitors have shown clinical efficacy but they often suffer from two major drawbacks: low specificity caused by the structural similarity among the ATP-binding sites and the acquired resistance caused by multiple mechanisms, including mutation(s) of the target kinases and activation of alternate signaling pathways²⁷. As provided herein, the targeting of lipid binding of SH2 domains of kinases was investigated. SH2 domains bind lipids using highly variable binding sites²¹ and SH2-lipid binding controls not only the enzymatic activity of kinases but also their scaffolding function²¹⁻²³ which has been implicated in the mechanism of drug resistance but cannot be blocked by conventional kinase inhibitors^{28,29}. Not wishing to be bound by theory, targeting lipid binding of SH2 domains was used to develop specific, potent, and resistance-defying kinase inhibitors.

Spleen tyrosine kinase (Syk) is involved in various cell signaling pathways in hematopoietic cells, including immune myeloid and B cells³⁰. B cell receptor (BCR) signaling plays crucial roles in adaptive immunity and hematologic malignancies³¹⁻³³ Most BCR signaling proteins, including Syk, B-cell linker (BLNK), phospholipase Cγ2 (PLCγ2), Bruton's tyrosine kinase (Btk), and phosphoinositide 3-kinase δ (PI3Kδ), contain a (or more) lipid-binding SH2 domain coordinating complex protein-protein interactions. Among BCR signaling proteins, Syk, Btk, and PI3Kδ have been primary drug targets for B cell malignancies³⁴. In particular, kinase inhibitors of Btk (e.g., ibrutinib) and PI3Kδ (e.g., idelalisib) have shown strong clinical activities in diverse B cell malignancies, including chronic lymphoid leukemia (CLL)³⁴.

Since Syk also plays a key role in regulation of myeloid cells, Syk inhibitors were thought to find broader applications to hematologic malignancies than other inhibitors³⁰. A number of small molecule ATP-competitive inhibitors of Syk, such as fostamatinib (R788), cerdulatinib (PRT062070), and TAK-659, have thus been developed and evaluated³⁵. However, they did not produce promising results in the clinical trials due to low specificity and significant cytotoxicity³⁵. Entospletinib is a second generation Syk inhibitor with higher Syk specificity that showed a promise in treating hematologic malignancies, including acute myeloid leukemia (AML) and CLL³⁶. However, it has been reported that these cells rapidly acquire drug resistance during the treatment with entospletinib, rendering it ineffective³⁷. Therefore, development of a new type of resistance-defying or -suppressing Syk inhibitor is still an unmet need.

Small molecule inhibitors for kinases are the mainstay of targeted cancer therapy but drug resistance remains a major problem in conventional kinase inhibitor development. The compounds described herein address this challenge. In one aspect, the compounds described herein target lipid-protein interactions that are essential for cellular functions of numerous non-receptor kinases containing the Src homology 2 (SH2) domain. Recent studies have shown that membrane lipids control both catalytic and non-catalytic scaffolding activity of these kinases via lipid-SH2 domain interaction. Here we report development of novel non-lipidic small molecule inhibitors of the lipid-SH2 domain interaction that potently and specifically block the cellular activity of their host proteins.

In one aspect, described herein are compounds that are inhibitors for spleen tyrosine kinase (Syk), which is a key signaling protein in hematologic cells and implicated in multiple hematopoietic malignancies, cancer (e.g., chronic lymphoid leukemia (CLL) or acute myeloid leukemia (AML)), diabetes, and immune disorders. In one aspect, the compounds described herein drug resistance, which renders current ATP-competitive Syk inhibitors ineffective.

### Aspects

Aspect 1. A compound of formula I, or a pharmaceutically acceptable salt thereof

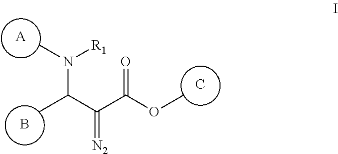

wherein

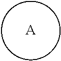

is 5 or 6 membered substituted or unsubstituted heteroaryl;

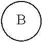

is 5 to 10 membered substituted or unsubstituted heteroaryl or substituted or unsubstituted phenyl;

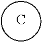

is cycloalkyl; and
R₁ is H or C₁-C₄ alkyl.

Aspect 2. The compound of Aspect 1, wherein

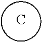

is cyclohexyl.

Aspect 3. The compound of Aspect 1 or 2, wherein

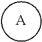

is a 6-membered substituted or unsubstituted heteroaryl.

Aspect 4. The compound of Aspect 1 or 2, wherein

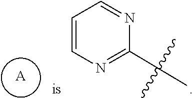

Aspect 5. The compound in any one of Aspects 1-4, wherein R¹ is H.

Aspect 6. The compound in any one of Aspects 1-5, wherein

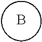

is a 5 or 9-membered substituted or unsubstituted heteroaryl.

Aspect 7. The compound in any one of Aspects 1-5, wherein

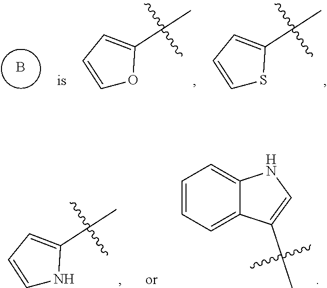

Aspect 8. The compound of Aspect 1, wherein

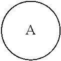

is a 6-membered substituted or unsubstituted heteroaryl,

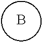

is a 5 membered substituted or unsubstituted heteroaryl or a 9 membered fused heteroaryl,

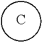

is cyclohexyl, and
R¹ is hydrogen.

Aspect 9. The compound of Aspect 1, wherein the compound is formula II

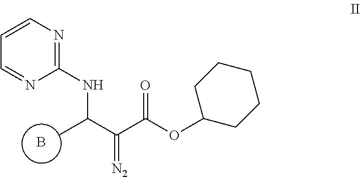

Aspect 10. The compound of Aspect 9, wherein

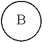

is a 5 membered substituted or unsubstituted heteroaryl.

Aspect 11. The compound of Aspect 10, wherein the 5 membered heteroaryl is a substituted or unsubstituted furan, a substituted or unsubstituted pyrrole, or a substituted or unsubstituted thiophene.

Aspect 12. The compound of Aspect 10, wherein the 5 membered heteroaryl is an unsubstituted furan or an unsubstituted pyrrole.

Aspect 13. The compound of Aspect 9, wherein

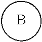

is a 9 membered fused substituted or unsubstituted heteroaryl.

Aspect 14. The compound of Aspect 13, wherein the 9 membered substituted or unsubstituted fused heteroaryl is a substituted or unsubstituted indole, a substituted or unsubstituted isoindole, a substituted or unsubstituted indolizine, a substituted or unsubstituted purine, or a substituted or unsubstituted indole.

Aspect 15. The compound of Aspect 13, wherein the 9 membered substituted or unsubstituted fused heteroaryl is an unsubstituted indole.

Aspect 16. The compound of Aspect 1, wherein the compound is

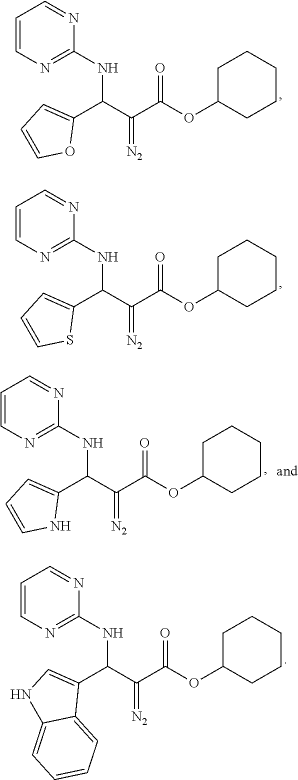

Aspect 17. A pharmaceutical composition comprising a compound of any one of Aspects 1 to 16 and a pharmaceutically acceptable carrier.

Aspect 18. A method of treating a subject suffering from a disease or disorder for which inhibiting spleen tyrosine kinase (Syk) would provide a benefit comprising administering to the subject an effective amount of the compound of any one of Aspects 1 to 16, or a pharmaceutically acceptable salt thereof.

Aspect 19. The method of Aspect 18 wherein the disease or disorder is selected from cancer, diabetes, and immune disorders.

Aspect 20. The method of Aspect 18, wherein the disease or disorder is selected from chronic lymphoid leukemia (CLL) or acute myeloid leukemia (AML).

Aspect 21. A method of inhibiting spleen tyrosine kinase (Syk) in a cell comprising contacting the cell with an effective amount of any one of Aspects 1 to 16, or a pharmaceutically acceptable salt thereof.

Aspect 22. A method of treating chronic lymphoid leukemia (CLL) or acute myeloid leukemia (AML) in a subject comprising administering to the subject an effective amount of the compound of any one of Aspects 1 to 16, or a pharmaceutically acceptable salt thereof.

Aspect 23. A compound of formula I, or a pharmaceutically acceptable salt thereof

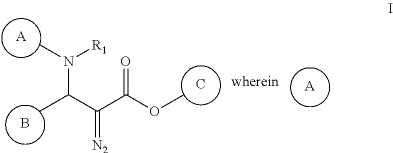

is 5 or 6 membered substituted or unsubstituted heteroaryl;

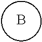

is 5 to 10 membered substituted or unsubstituted heteroaryl or substituted or unsubstituted phenyl;

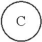

is cycloalkyl; and
R₁ is H or C₁-C₄ alkyl.

Aspect 24. The compound of Aspect 23, wherein

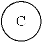

is cyclohexyl.

Aspect 25. The compound of Aspect 23 or 24, wherein

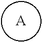

is a 6-membered substituted or unsubstituted heteroaryl, preferably

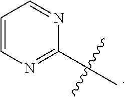 [SMILES: *c1ncccn1]

Aspect 26. The compound in any one of Aspects 23-25, wherein R¹ is H.

Aspect 27. The compound in any one of Aspects 23-25, wherein

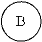

is a 5 or 9-membered substituted or unsubstituted heteroaryl, preferably

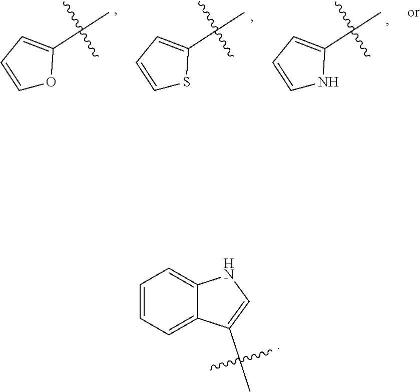 [SMILES: *c1ccco1 ; *c1cccs1 ; *c1ccc[nH]1 ; *c1c[nH]c2ccccc12]

Aspect 28. The compound of Aspect 23, wherein

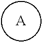

is a 6-membered substituted or unsubstituted heteroaryl,

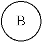

is a 5 membered substituted or unsubstituted heteroaryl or a 9 membered fused heteroaryl,

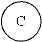

is cyclohexyl, and
R¹ is hydrogen.

Aspect 29. The compound of Aspect 1, wherein the compound is formula II

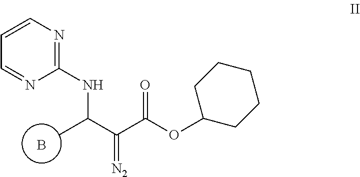

Aspect 30. The compound of Aspect 29, wherein

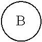

is a 5 membered substituted or unsubstituted heteroaryl, preferably a substituted or unsubstituted furan, a substituted or unsubstituted pyrrole, or a substituted or unsubstituted thiophene.

Aspect 31. The compound of Aspect 29, wherein

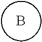

is a 9 membered fused substituted or unsubstituted heteroaryl, preferably a substituted or unsubstituted indole, a substituted or unsubstituted isoindole, a substituted or unsubstituted indolizine, a substituted or unsubstituted purine, or a substituted or unsubstituted indole.

Aspect 32. The compound of Aspect 23, wherein the compound is

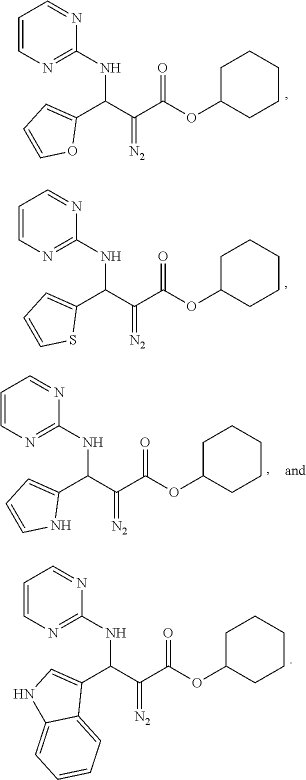

Aspect 33. A pharmaceutical composition comprising a compound of any one of Aspects 23 to 33 and a pharmaceutically acceptable carrier.

Aspect 34. A method of treating a subject suffering from a disease or disorder for which inhibiting spleen tyrosine kinase (Syk) would provide a benefit comprising administering to the subject an effective amount of the compound of any one of Aspects 23 to 33, or a pharmaceutically acceptable salt thereof.

Aspect 35. The method of Aspect 34 wherein the disease or disorder is selected from cancer, diabetes, and immune disorders, preferably chronic lymphoid leukemia (CLL) or acute myeloid leukemia (AML).

Aspect 36. A method of inhibiting spleen tyrosine kinase (Syk) in a cell comprising contacting the cell with an effective amount of any one of Aspects 23 to 33, or a pharmaceutically acceptable salt thereof.

Aspect 37. A method of treating chronic lymphoid leukemia (CLL) or acute myeloid leukemia (AML) in a subject comprising administering to the subject an effective amount of the compound of any one of Aspects 23 to 33, or a pharmaceutically acceptable salt thereof.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### Materials and Methods

### Materials

1-Palmitoyl-2-oleoyl-sn-glycero-3-phosphocholine (POPC) and 1-palmitoyl-2-oleoyl-sn-glycero-3-phosphoserine (POPS) were purchased from Avanti Polar Lipids. 1,2-dipalmitoyl derivatives of phosphatidylinositol-4,5-bisphosphate (PI(4,5)P₂), phosphatidylinositol-3,4,5-bisphosphate (PIP₃), and other phosphoinositides were from Cayman Chemical Co. The custom designed peptide was purchase from AlanScientific. shRNA's for human Syk were purchased from Integrated DNA Technologies and the transfection reagent JetPRIME was from Polyplus transfection. Antibodies against phospho-Syk (pS465/pS467), STAT3, phospho-STAT3 (pY705), STAT5, phospho-STAT5 (pY694), ERK1/2, and phospho-ERK1/2 (pT202/Y204) were purchased from Cell Signaling Technologies. Syk and BLNK antibodies were from Santa Cruz Biotechnology. The GAPDH antibody was from Sigma-Aldrich. Entospletinib and GDC-0941 were purchased from MedChemPress and Selleckchem, respectively. Jurkat, MV4-11, and HL60 cells were purchased from ATCC.

### Protein Expression and Purification

All SH2 domain constructs were cloned into the pRSET-B vector, were expressed as soluble proteins in Escherichia coli BL21 (DE3) pLysS (Novagen) cells with an N-terminal polyhistidine and a C-terminal EGFP tag. Cells were grown at 37° C. in 2 L of Luria broth containing either 100 μg/ml ampicillin or 50 μg/ml kanamycin, and protein was induced with 1 mM isopropyl 1-thio-β-D-galactopyranoside at 18° C. for 12 h when the OD600 reached ˜0.6. Cells were harvested by centrifugation at 4000×g at 4° C., and the pellets were stored at −80° C. Frozen pellets were thawed and resuspended in ice-cold 50 mM Tris buffer, pH 7.9, 300 mM NaCl, 10 mM imidazole, 10% (v/v) glycerol, 1 mM dithiothreitol, and 1 mM phenylmethylsulfonyl fluoride. The suspension was sonicated on ice to disrupt cells. After centrifugation at 14,000×g for 15 min at 4° C., an insoluble pellet was discarded and the clear supernatant was filtered using a 0.45-μm syringe filter. The supernatant was gently shaken with 2 ml of Ni-NTA resin (50% slurry) (Marvelgent Biosciences) at 4° C. for 1 h, and the resin was then packed into a column. The resin was washed with 50 ml of buffer containing 50 mM Tris, 160 mM NaCl, and 40 mM imidazole, pH 7.4. The protein was eluted with 10 ml of buffer containing 50 mM Tris, 160 mM NaCl, and 300 mM imidazole, pH 7.4. Protein was stored at 4° C.

### Synthesis of Syk Inhibitors

An oven dried flask containing a magnetic stirrer bar was charged with 2-aminopyrimidine (104.5 mg, 1.1 mmol), an aldehyde (1 mmol), 4A activated molecular sieves (125 mg), and yttrium(III) trifluoromethanesulfonate (56.8 mg, 0.1 mmol) under N₂ atmosphere. Dry dichloromethane (2.5 mL) was added, and the mixture was stirred 10 min at room temperature and then cooled to 4° C. c-Hex-diazoacetate⁷² (201.6 mg, 1.2 mmol) in dichloromethane (1 mL) was added, and the reaction mixture was stirred at this temperature and monitored by the thin layer chromatography until complete conversion. The resulting mixture was filtered on Celite and concentrated under a reduced pressure. The crude was purified by column chromatography on a silica gel (hexane/ethylacetate) to afford the corresponding diazo-compounds, WC35, WC36, and WC38. These compounds were tested for their biological activities as racemic mixtures.

### Cyclohexyl 2-diazo-3-(furan-2-yl)-3-(pyrimidin-2-ylamino)propanoate (WC35)

57% yield; ¹H NMR (500 MHz, CDCl₃): δ 8.29 (d, J=4.6 Hz, 2H), 7.37 (s, 1H), 6.60-6.63 (m, 1H), 6.33-6.38 (m, 3H), 6.22-6.25 (m, 1H), 4.83-4.88 (m, 1H), 1.79-1.81 (m, 2H), 1.64-1.66 (m, 2H), 1.24-1.47 (m, 4H); ¹³C NMR (125 MHz, CDCl₃): δ 161.1, 158.1, 151.7, 142.4, 111.9, 110.5, 1-7.0, 46.6, 31.7, 31.6, 25.3, 23.5; HRMS (ESI): calculated for C₁₇H₂₀N₅O₃ [M+H]⁺ 342.1566, found 342.1568.

### Cyclohexyl 2-diazo-3-(1H-indol-3-yl)-3-(pyrimidin-2-ylamino)propanoate (WC36)

55% yield; ¹H NMR (500 MHz, CDCl₃) 10.67 (d, J=12 Hz, 1H), 8.43 (d, J=4.7 Hz, 2H), 8.24-8.30 (m, 2H), 7.70 (d, J=7.85 Hz, 1H), 7.34 (d, J=8 Hz, 1H), 7.12-7.22 (m, 3H), 6.79 (t, J=4.75 Hz, 1H), 4.97-5.02 (m, 1H), 1.90-1.93 (m, 2H), 1.64-1.68 (m, 2H), 1.33-1.53 (m, 6H); ¹³C NMR (125 MHz, CDCl₃) δ 168.6, 158.3, 138.9, 135.9, 127.3, 127.3, 121.8, 120.2, 119.5, 114.4, 112.9, 111.2, 100.5, 72.8, 31.7, 25.4, 23.8; HRMS (ESI) calculated for C₂₁H₂₃N₆O₂ [M+H]⁺ 391.1882, found 391.1886.

### Cyclohexyl 2-diazo-3-(pyrimidin-2-ylamino)-3-(1H-pyrrol-2-yl)propanoate (WC38)

50% yield; ¹H NMR (500 MHz, CDCl₃): δ 8.89 (d, J=2.35 Hz, 2H), 8.16 (s, 1H), 7.71-7.73 (m, 1H), 7.37-7.40 (m, 1H), 6.97-6.98 (m, 1H), 6.69-6.71 (m, 1H), 5.00-5.07 (m, 1H), 1.95-1.99 (m, 2H), 1.76-1.79 (m, 2H), 1.32-1.60 (m, 6H); ¹³C NMR (125 MHz, CDCl₃): δ 163.3, 159.0, 158.0, 145.1, 130.5, 127.0, 120.2, 116.6, 115.7, 108.3, 106.5, 73.8, 31.7, 25.4, 23.8; HRMS (ESI): calculated for C₁₇H₂₁N₆O₂ [M+H]⁺ 341.1726, found 341.1729.

### Synthesis of Biotinylated WC36 (WC36B; N-(2-(2-diazo-3-(1H-indol-3-yl)-3-(pyrimidin-2-ylamino)propanamido)ethyl)-5-((3aR,4R,6aS)-2-oxohexahydro-1H-thieno[3,4d]imidazol-4-yl)pentanamide)

Flask containing a magnetic stirrer bar was charged with WC-36 (195 mg, 0.5 mmol), and biotinamide (121.5 mg, 0.5 mmol) under N₂ atmosphere. Methanol (5 mL) and triethylamine (100 ml) was added, and the mixture was stirred overnight at the room temperature. The resulting mixture was concentrated under a reduced pressure. The crude was purified by column chromatography on a silica gel (hexane/ethylacetate) to afford the corresponding WC36B. 42% yield; ¹H NMR (500 MHz, CDCl₃): δ 9.71 (brs, 1H), 8.20-8.23 (m, 2H), 7.47-7.50 (m, 1H), 7.26-7.33 (m, 2H), 6.98-7.10 (m, 3H), 6.78-6.83 (m, 1H), 6.60-6.67 (m, 1H), 6.54 (t, 1H, J=4.70 Hz), 6.03 (brs, 1H), 5.50-5.52 (m, 2H), 4.76-4.98 (m, 2H), 4.02-4.21 (m, 2H), 2.89-3.22 (m, 4H), 2.50-2.67 (m, 2H), 1.27-2.01 (m, 12H). ¹³C NMR (125 MHz, CDCl₃): δ 174.1, 169.8, 168.8, 164.5, 163.1, 158.2, 153.0, 148.6, 136.3, 136.0, 127.6, 126.8, 125.0, 122.8, 121.5, 121.3, 120.2, 119.1, 118.8, 111.4, 111.3, 108.4, 93.2, 90.5, 71.6, 61.8, 60.2, 55.6, 47.5, 40.3, 35.8, 35.6, 32.1, 31.9, 28.1, 27.9, 25.5, 23.9; HRMS (ESI): calculated for C₂₇H₃₃N₁₀O₃S [M]+ 577.2630, found 577.2645.

### Lipid Vesicle Preparation

Large unilamellar vesicles (LUVs) with 100-nm diameter were prepared by mixing the lipid solutions in chloroform according to the final lipid composition and the solvent was evaporated under the gentle stream of nitrogen gas. 20 mM Tris buffer, pH 7.4, containing 0.16 M NaCl was added to the lipid film to adjust the final lipid concentration, and the mixture was vortexed for 1 min, and sonicated in a sonicating bath for 1 min to break multilamellar vesicles. LUVs were prepared by multiple extrusion through a 100-nm polycarbonate filter (Avanti) using a Mini Extruder (Avanti).

### Surface Plasmon Resonance Analysis

All SPR measurements were performed at 23° C. in 20 mM Tris, pH 7.4, containing 0.16 M NaCl using a lipid-coated L1 chip in the BIACORE X-100 system (GE Healthcare) as described previously^{44,73}. LUVs of POPC/POPS/PtdInsP (77:20:3) and POPC were used as the active surface and the control surface, respectively. Sensorgrams were collected for both membrane association and dissociation but the only the association phases were further analyzed because the dissociation phases were often too slow for analysis. All steady-state SPR measurements were carried out for more than 10 different concentrations within a 10-fold range of the K_{d} of the proteins, while maintaining the slow flow rate of injection of protein (i.e., at 5-10 μl/min) to provide enough time to reach the RU values of the association phase to the nearest its equilibrium value (R_{eq}). For K_{d} determination, sensorgrams obtained at varying protein concentrations were analyzed assuming a Langmuir-type binding between the protein (P) and protein binding sites (M) on vesicles (that is, P+M↔PM). The R_{eq} values were plotted against the protein concentrations (Pₒ), and the K_{d} was established by nonlinear least squares analysis of the binding isotherm using the equation, R_{eq}=Rₘₐₓ/(1+K_{d}/Pₒ) where Rₘₐₓ indicates the maximal R_{eq} value. The average and standard deviation values of K_{d} were obtained from three or more measurements.

### Fluorescence Plate Reader Assay and Small Molecule Inhibitor Screening

The membrane-binding assay is based on fluorescence quenching of EGFP fused to a SH2 domain by a dark quencher containing lipid, dabsyl-PE, incorporated in lipid vesicles. The plate reader assay was performed using the Synergy™ Neo spectrofluorometer at 25° C. Nontreated black polystyrene 96-well plates (Corning) were used. Protein (10-50 nM) with the increasing concentration of vesicles with a given lipid composition in 20 mM Tris buffer, pH 7.4, containing 0.16 M NaCl was added to each row of wells and the EGFP fluorescence emission at 516 nm was measured with excitation set at 485 nm. Z′ factor for the assay system was determined as described⁷⁴. Iₘₐₓ and IC₅₀ values of inhibitors were determined using the equation, I=Iₘₐₓ/(1+IC₅₀/[I]) where I and [I] indicate % inhibition and inhibitor concentration, respectively. The average and standard deviation values were obtained from triplicate determinations.

### Fluorescence Anisotropy Assay for SH2-pY Peptide Binding

The fluorescein-6-aminohexanoyl (F-Ahx)-labeled Igα peptide (F-Ahx-YDMTTpSGpSGpSGLPLL) was dissolved in dimethyl sulfoxide to yield 1 mg/ml stock solution. The peptide solution was diluted to 1-10 μM with 20 mM Tris buffer, pH 7.9, containing 160 mM NaCl for binding studies. 300 μl of the Syk-cSH2 (WT or mutants) solution (0-250 μM) was added to a series of 1.5 ml microcentrifuge containing the peptide solution (2.5 μM). After 10-min incubation in the dark, the mixture was transferred to a quartz cuvette with 2-mm path length and fluorescence anisotropy (r) was measured with excitation and emission wavelengths set at 485 and 535 nm, respectively using Horiba Flurolog-3 spectrofluorometer. Since Pₒ>>Pepₒ under our conditions, the K_{d} for the Syk-cSH2-peptide binding was determined by the non-linear least-squares analysis of the binding isotherm using the equation:

$${Pep}_{bound} / {Pep}_{0} = Δ r / Δ {r}_{m ax} = \frac{1}{1 + {k}_{d} / {P}_{0}}$$

where Pep_{bound}, Pepₒ, and Pₒ indicate the concentration of bound peptide, total peptide and total Syk-cSH2, respectively, and Δr and Δrₘₐₓ are the anisotropy change for each Pₒ and the maximal Δr, respectively.

### Cell Culture

DT40 cell WT and DT40-Syk^{-/-} cells were purchased from Riken, Japan. Both cell types were maintained in the Gibco RPMI 1640 medium (ThermoFisher), supplemented with 10% heat-inactivated fetal bovine serum (FBS) (Sigma), 1% chicken serum (Sigma), 50 μM β-mercaptophenol (ThermoFisher) and 1% penicillin/streptomycin (ThermoFisher). Jurkat cells were maintained in the RPMI 1640 medium supplemented with 10% heat-inactivated FBS, 1 mM HEPES, and 1% penicillin/streptomycin. MV4-11 AML cells were maintained in the Gibco IMDM medium (ThermoFisher), supplemented with 10% heat-inactivated FBS. HL-60 AML cells were maintained in IMDM supplemented with 20% heat-inactivated FBS.

### Cell Transfection

DT40-Syk^{-/-} cells were maintained at a cell density of 5.0×10⁶ cells/ml before transfection. Cells harvested by centrifugation were resuspended in 100 μl Nucleofector T-Kit containing 3 μg of EGFP-Syk WT (or K220/K222A) and 2 μg of mCherry-mCherry-eMyoX-tPH in a microcentrifuge tube. The mixture was then transferred to the cuvette provided with the kit and cells were electroporated by placing the cuvette in the nucleofector 2b platform (Lonza) and selecting the program B-009. Immediately after the electroporation, 500 μl of the growth media was added and the mixture was transferred in 100 mm plate. MV4-11 cells were transfected with different siRNA's (300 pmol) for Syk knockdown using the Nucleofector L-kit and program Q-023. After 72 h of transfection, the cells were activated using IgG2 (Sigma-Aldrich) to stimulate the human Fc-γ receptor I and harvested for western blot analysis.

### Subcellular Localization Analysis

DT40-Syk^{-/-} cells transfected with EGFP-Syk WT (or K220/K222A) and mCherry-mCherry-eMyoX-tPH were imaged under 100× magnification with a Nikon Eclipse Ti2 microscope using the NIS element software. For IgM stimulation, cells were dropped on the IgM-coated surface (10 ng/ml) covered with the imaging buffer (HBSS pH 7.4, 2 mM MgCl₂, 1% FBS) and incubated for 10 min before imaging. Pearson's correlation coefficient (y) was calculated using Coloc 2, which is a Fiji's plugin for colocalization analysis. The plasma membrane was selected as the region of interest for calculation of the coefficient. Averages ±s.d.'s of γ values were calculated from triplicate determinations.

### Cellular Calcium Assay

Ca²⁺ flux in DT40 cells was measured under 40× magnification with a Nikon Eclipse Ti2 microscope and accompanying NIS element software. Briefly, cells were loaded with 5 μM Fura-2 AM (ThermoFisher) in the presence of 5 μM Pluronic F-127 (Sigma-Aldrich) in Hank's balanced salt solution (HBSS: ThermoFisher) for 45 min at room temperature. Cells were washed three times with HBSS and kept for an additional 30 min at room temperature before the Ca²⁺ measurement. Cells were dropped on the IgM-coated surface (10 ng/ml) covered with the imaging buffer (HBSS pH 7.4, 2 mM MgCl₂, 1% FBS), and the ratiometric images (F₃₄₀/F₃₈₀: ratio of fluorescence emission intensity at 340/380 nm) were acquired every 30 msec for 10 min. All quantifications were performed using the Fiji software.

### Co-Immunoprecipitation

MV-4-11 cells were collected by centrifugation, washed twice with phosphate phosphate-buffered saline (PBS) and resuspended in the immunoprecipitation (IP) buffer (50 mM HEPES, pH 7.4, 150 mM NaCl, 1 mM MgCl₂, 1 mM EGTA, and 0.25 mM GTP) with 0.2-0.5% Triton X-100 and a protease inhibitor mix (Sigma-Aldrich). Lysates were prepared after 5 min of incubation on ice by repeated pipetting and centrifugation for 10 min at 12,000 g and at 4° C. For immunoprecipitation, the SYK-specific antibody was immobilized onto 100 μl Dynabeads/protein A (ThermoFisher) and the excess antibody was washed away by placing the tube in a DynaMag magnet and removing the supernatant. The resuspended beads were then incubated with the cell extract supernatant containing 500 μg of proteins for 1 h at 4° C. The immunoprecipitates were rinsed three times with the IP buffer and eluted with 30 μl of the sample buffer containing sodium dodecyl sulfate (SDS). For detection of proteins in total extracts (i.e., Input), 30 μg of the sample was loaded in each gel lane for western blot analysis.

### Streptavidin Pull Down Assay

Streptavidin-coated beads (Dynabeads™ M280 streptavidin beads; ThermoFisher) were washed twice with 1 ml of PBST (PBS+0.1% Tween 20) and resuspended in 100 μl of PBST. WC36B (or biotin) (10 mM) was added to 100 μl of the bead suspension and the mixture was incubated at room temperature for 15 min with gentle shaking. The mixture was then vortexed for 5 s and the tube was placed on a magnet for 1 min. After discarding the supernatant, the beads were resuspended in the same volume of the washing buffer. Washing is repeated three times to remove excess WC36B. WC36B-coated beads were then incubated with the cell lysate (MV-4-11) for 30 min at room temperature with gentle shaking. Protein-coated beads were separated with a magnet for 2-3 min and washed 4-5 times with PBS containing 0.1% BSA. The protein was then removed from the beads by boiling the beads in the SDS-polyacrylamide gel electrophoresis (PAGE) application buffer. Samples were concentrated for the mass spectrometry analysis and the western blot assay.

### Western Blot Analysis

Cells were seeded at a density of 3.0×10⁵ cells/well prior to incubation with inhibitors. Cells were incubated with 1-5 μM of Syk inhibitors overnight and stimulated with different antibodies: IgG2 for AML cells, IgM for Raji B cells, and OKT3 (Biolegend) for Jurkat cells. The cells were lysed in NP40 lysis buffer (50 mM Tris, pH 7.5, 150 mM NaCl, 1 mM EDTA, 1% NP40, 10% glycerol, 10 mM NaF, 10 mM Na₃VO₄, and the protease inhibitor cocktail) at 4° C. and the cell debris was removed by centrifugation. Samples were analyzed by SDS-PAGE. Proteins were separated and transferred to Immobilon-P polyvinylidene difluoride membrane (Millipore Sigma). The membrane was blocked with 5% bovine serum albumin for 1 h and incubated overnight at 4° C. with various antibodies (1:1000 dilution for all antibodies). After the unbound antibodies were removed by washing with 0.1% Tris buffer saline with 0.1% Tween20, the membranes were incubated with the horseradish peroxidase secondary antibody (1:5000 dilution) for 1 h at room temperature. The membranes were washed three more times with 0.1% Tris buffer saline with 0.1% Tween20 to remove the unbound horseradish peroxidase secondary antibody before imaging. The chemiluminescence intensity of protein bands in the gel was analyzed and documented by the Azure 500Q Imaging System.

### Generation of Entospletinib (or WC36)-Resistant Cells

MV4-11 cells were treated with entospletinib (or WC36) to induce drug-resistance cells as reported previously³⁷. MV4-11 cells were incubated first with 500 nM entospletinib (or WC36) for the first week, then the drug concentration was increased 0.5 μM per week until it reached 5 μM (i.e., 10 weeks). Then the concentration was maintained at 5 μM. When the cells remained >90% viable in the presence of 5 μM (i.e., 10×IC₅₀) entospletinib, they were considered entospletinib-resistant.

### Cell Proliferation Assay

MV4-11 or HL-60 cells were seeded into a clear-bottom 96-well plate containing varying concentrations of inhibitors in the optimal growth media described above and the mixtures were incubated for 16 h. Cells were then treated with a mixture of XTT-labeling reagents and the electron coupling reagents according to the manufacturer's protocol (Roche). After 4 h, the absorbance values at 475 nm and at 660 nm were simultaneously measured by Synergy™ Neo spectrofluorometer at 25° C. Cells with the growth media was used for background correction.

### Isolation and Treatment of Patient-Derived AML Cells.

Primary samples were obtained from an existing HEMBANK at UIC under an IRB approved research protocol (protocol #2021-0723). Cells were processed and treated with inhibitors as reported previously⁷⁵. For primary AML samples, mononuclear cells isolated by Ficoll density centrifugation were utilized. mononuclear cells were treated in culture at a density of 0.5×10⁶/ml in StemSpan with CD34 expansion supplement (Stemcell Technologies) for 24 h and then harvested for immunoblotting assays. In parallel 2×10⁴ cells were plated in triplicate for each treatment condition in Methoccult H4434 (Stem Cell Technologies) in the presence of drug. At days 12-14, colonies were manually enumerated under light microscopy and normalized to DMSO control for each individual sample due to interpatient variability in colony forming activity. Normalized colony forming units for pooled treated and untreated samples (n=4) were compared using unpaired 2-tailed t test. p<0.05 was considered statistically significant.

### Cell Viability Assay

All cell viability assays were conducted in a white, flat-bottom, 96-well tissue culture plate (Corning). The Raji cells were seeded at a density of 3.0×10⁵ cells/well 24 h prior to the incubation with the inhibitor. Varying concentrations of WC36 (or DMSO) was then added to each well and the plates were incubated at 37° C. and at 5% CO₂ for 72 h. Cell viability was quantified with the CellTiter-Glo reagent. Growth media were removed by aspiration and replaced with 100 μl fresh media and 100 μl CellTiter-Glo reagent. The plates were then gently shaken for 2 min and incubated at 25 C.° for additional 8 min. The luminescence was measured using Synergy™ Neo spectrofluorometer at 25 C.°. The luminescence value at each WC36 concentration was normalized against that without WC36. DMSO with the same volume as the WC36 solution was used as negative controls.

### Molecular Dynamics Simulation System Setup

The simulation system for each SH2 domain (Syk-cSH2, BLNK-SH2, and PLCγ2-cSH2) was constructed using the NMR structure of Syk-cSH2 (PDB: 1CSY), the NMR structure of BLNK-SH2 (PDB: 2EO6), and a homology model of PLCγ2-cSH2 built using the crystal structure of C-terminal SH2 domain of PLCγ1 (PDB: 4EY0) as the template, respectively. The homology modeling was carried out using Prime in the Schrödinger Suite (release 2019 April). PSFGEN plugin of VMD (Visual Molecular Dynamics)⁷⁶ was employed to add a C-terminal carboxylate capping group, an N-terminal ammonium capping group, and hydrogen atoms.

For ensemble docking of compounds, each SH2 domain was solvated in a TIP3P water box with a 20 Å padding and neutralized with 150 mM NaCl. To capture the membrane association phenomenon of SH2 domains, we performed membrane-binding simulations by employing a highly mobile membrane mimetic (HMMM) model described as below.

All the independent HMMM membranes were constructed using HMMM BUILDER in CHARMM-GUI⁷⁷. Due to the presence of short-tailed lipids and organic solvent, DCLE, which mimics the hydrophobic core of the membrane, HMMM models significantly enhance lipid diffusion and membrane reorganization thereby allowing spontaneous insertion of peripheral protein. This approach has been extensively used to study variety of peripheral and integral membrane proteins With the aid of this accelerated membrane model, we were able to perform multiple membrane-binding simulations of each SH2 domain in the presence of mixed lipid membrane containing 1,2-dihexanoyl derivatives of PC, PS, and PIP₃ in the ratio of 74:20:6. Considering the possible protonation states of PIP₃, three variants of PIP₃ headgroup were employed in the simulations. Out of six PIP₃ in each leaflet, two PIP₃ molecules were protonated at P3 position, two at P4 position, and the other two at P5. The entire membrane/protein system was solvated with TIP3P water and buffered in 150 mM NaCl to keep the system neutral.

All the membrane-binding simulations started by placing each SH2 domain in the aqueous solution at least 10 Å away from the membrane. To ensure the final membrane-bound conformation of the protein is not biased due to its initial placement, we generated different initial configurations by varying the orientation of each SH2 domain with respect to the membrane normal (Z axis).

### Molecular Dynamics Simulation Protocols

All molecular dynamics simulations were performed in NAMD2⁷⁸ employing CHARMM36m protein and lipid force fields^{79,80} and TIP3P water. Non-bonded interactions were calculated with 12 Å cutoff and a switching distance of 10 Å. Long-range electrostatic interactions were calculated using the particle mesh Ewald (PME) method⁸¹. The temperature was maintained at 310 K by Langevin dynamics with a damping coefficient of 1.0 ps⁻¹. For the ensemble docking systems, NPT ensemble was used with the Nose-Hoover Langevin piston method⁸². In HMMM simulations, short tailed HMMM lipid membranes were simulated with a constant x-y area corresponding to a 10% increase in the average area per lipid to enhance lipid lateral diffusion and protein insertion to the membrane. The pressure was therefore maintained at 1 atm only along the membrane normal (NPAT) using the Nosé-Hoover Langevin piston method⁸². All the simulations were performed with a 2 fs timestep.

All constructed systems were first energy minimized for 10,000 steps by employing steepest descent algorithm. For the systems prepared for ensemble docking, each SH2 domain was equilibrated for 300 ns. For all membrane systems, the energy minimized system was then simulated for 10 ns with harmonic restraints (k=0.5 kcal mol⁻¹ Å⁻²) on Cα atoms of the protein, followed by a restraint-free production run of 100 ns. To prevent the escape of the short-tail lipids into the aqueous phase, a harmonic restraint with a force constant of k=0.05 kcal mol⁻¹ Å⁻² was applied to the phospholipid tail atoms C21 C31, along the Z axis of the simulation box. To prevent the diffusion of the DCLE molecules out of the membrane core, we subjected them to a grid-based restraining potential, applied using the gridForce⁸³ module of NAMD⁸⁴.

### Ensemble Docking

To identify a potential drug binding site in each SH2 domain, the Schrödinger Suite (release 2019 April) was used for docking each compound to an ensemble of the SH2 structures extracted from the equilibrium MD simulation. Each inhibitor molecule was first prepared and generated using the LigPrep with OPLS3e forcefield⁸⁵. We extracted protein snapshots for every ns from the 300-ns equilibrated simulation to account for protein structural dynamics, totaling 300 protein snapshots. The grid files for each protein snapshot were generated using the cross docking XGlide script (xglide.py) in the Schrödinger Suite, by which Protein Preparation Wizard⁸⁶ was first called to prepare and refine all protein structures, SiteMap⁸⁷ was then performed to identify potential ligand binding sites and set up the grid center accordingly, and the OPLS3e forcefield was used to generate the docking search grid. To dock each inhibitor to the grid files for each potential binding site, the Virtual Screening Workflow (VSW) in Maestro was used for carrying out Glide⁸⁸ extra precision docking runs with post-docking minimization. For each binding site, up to five poses were generated and the best scoring one was kept for each ligand state.

### Analysis of Syk-cSH2-PIP₃ Interactions

All the analysis was performed in VMD using in-house tcl scripts. The localization of Syk-cSH2 on the membrane surface, more-specifically its interaction with PIP₃ was gauged by monitoring the ensemble-averaged Z positions of all the residues (heavy atoms) with respect to the P4 plane, over the last 50 ns of HMMM membrane binding simulations. This analysis allowed us to understand which region of the SH2 domain comes close to the membrane and interacts with PIP₃. The total number of PIP₃ molecules that interact with the protein was monitored by counting any PIP₃ that localizes within 5 Å of the protein during the MD trajectories. As these coarse metrics may not provide sufficient details on the nature of protein-lipid interactions and the information of the binding site, we also quantified specific interactions between lipid headgroups and the SH2 domain. Based on our previous experience⁸⁹, a 3.5 Å heavy-atom cutoff was chosen to define lipid-protein contacts. We have calculated specific contacts between the protein and the phosphate groups at position 3, 4, and 5 of the inositol ring.

### Mass Spectrometry Analysis

MS analysis was performed to identify proteins captured by WC36B and biotin. Briefly, the eluant containing proteins from each capture were diluted to a final concentration of 5% SDS, reduced with 10 mM dithiothreitol at 55° C. for 15 min, alkylated with 30 mM iodoacetamide at room temperature for 20 min in the dark and enzymatically digested via trypsin at 37° C. overnight using the S-Trap protocol. Peptides from each capture were subsequently eluted, dried in vacuo and resuspended in 0.1% (v/v) formic acid. Peptide separation and mass detection occurred using an Agilent 1260 liquid chromatography (LC) system and Thermo Q-Exactive mass spectrometer. Raw data for the LC-MS analysis was searched against the Swiss Protein Homosapien database using the Proteome Discoverer (v2.3, Thermo Fisher, Carlsbad, CA) software. Here, trypsin was set as the protease with two missed cleavages and searches were performed with precursor and fragment mass error tolerances set to 10 ppm and 0.02 Da, respectively. Peptide variable modifications allowed during the search were oxidation (M), whereas carbamidomethyl (C) and was set as a fixed modification.

### Results

### SH2 Domains of BCR Signaling Proteins Specifically Bind PIP₃ Using Variable Sites

We previously reported that a large majority of SH2 domains bind lipids in the plasma membrane of the mammalian cells with high affinity²¹. Since many of them showed specificity for PtdInsPs²¹, we determined the PtdlnsP specificity of SH2 domains of Syk and two other BCR signaling proteins, BLNK and PLCγ2, by surface plasmon resonance (SPR) analysis. Syk and PLCγ2 have two SH2 domains but their C-terminal SH2 domains (cSH2) have much higher membrane affinity than N-terminal SH2 domains and we thus focused on the cSH2 domains in this study. SPR sensorgrams showed that all three SH2 domains preferentially bind phosphatidylinositol-3,4,5-trisphosphate (PIP₃) over other PtdInsPs. Selectivity of some of these SH2 domains among other PtdInsPs is slightly different from our reported data that were estimated from rapid screening analysis²¹.

Multiple biophysical studies have indicated that cytosolic signaling proteins bind the lipid membrane in various orientations and undergo significant conformational changes while interacting with the membrane⁷, However, the atomic-level mechanisms by which these proteins interact with lipids in the natural membrane environment remain elusive^{7,15,38}. This is mainly because current structural biology technologies do not allow for high-resolution structural determination of these cytosolic proteins bound to natural lipid bilayers. As a result, it is extremely difficult to rationally design small molecule inhibitors for lipid-protein interaction based on available structures of these proteins and validate the mode of their inhibitory actions by structural determination. Molecular dynamic simulations have been employed to address these problems but most current approaches either do not fully incorporate the essential features of the membrane-protein interaction or do not allow rapid survey of the conformational space¹⁵. To overcome the major obstacles, we applied to this study our advanced membrane-binding simulations using a highly mobile membrane mimetic (HMMM) membrane that allows for rapid lipid diffusion and spontaneous insertion of cytosolic proteins^{39,40}. Starting from the unbound aqueous-state configuration of Syk-cSH2, we simulated ten independent membrane-binding simulations of the protein in the presence of the HMMM membrane composed of 1,2-dihexanoyl derivatives of phosphatidylcholine (PC)/phosphatidylserine (PS)/PIP₃ (74:20:6). We quantified the interaction of the protein with PIP₃ by calculating the localization of each residue with respect to the phosphorylated inositol plane of PIP₃. Any protein residue close to this plane may have high propensity for PIP₃ binding. We consistently observed multiple interfaces through which the Syk-cSH2 domain interacts with PIP₃ molecules. Specifically, we captured higher propensity of H163, K165, K187, Y203, K220, K222, K232, K233, and K247 to interact with different phosphate moieties of PIP₃. Interestingly, we observed no interaction of PIP₃ with the pY-binding pocket (and its conserved cationic residue R195) of Syk-cSH2, suggesting that Syk-cSH2 can coincidently interact with the PIP₃-containing membrane and with pY-containing proteins.

To provide atomic-level information on the specific lipid-protein interactions between the residues of Syk-cSH2 and anionic PIP₃, we analyzed the last 50 ns of trajectories obtained from membrane-binding simulations. Overall, during these simulations, Syk-cSH2 remained associated with PIP₃ molecules via distinct hotspots. Among these hot spots, a shallow cationic cavity composed of H163, K165, Y203, K220, and K222 nicely accommodates the head group of a single PIP₃ molecule and make specific contacts with three phosphate moieties. In particular, K220 and K222 primarily form hydrogen bonds with the 3′-phosphate group, suggesting their essential role in PIP₃ specificity. The residues in other hot spots do not make specific contact with the 3′-phosphate group but rather non-specifically interact with other phosphate groups. For instance, K172, interacts with both 4′- and 5′-phosphate groups of PIP₃. Thus, their binding to PIP₃ is considered non-specific electrostatic interaction between the cationic membrane binding surface of Syk-SH2 and with anionic lipids, which does not contribute to PIP₃ specificity. It should be stressed that the local conformations of the putative PIP₃ binding cavity of Syk-cSH2 in the simulated structures are significantly different from that in the reported solution NMR structure⁴¹, again underscoring the difficulty in predicting the lipid binding site and designing inhibitors targeting the site using solution or crystal structures.

To experimentally validate the predicted PIP₃-binding site of Syk-cSH2, we performed mutational analysis. Specifically, we mutated K172, K220, and K222 to Ala. Since K172 is putatively involved in 4′ and 5′-phosphate binding, the K172A mutation should affect binding to PIP₃ as well as other 5-phosphoinositides, such as phosphatidylinositol-4,5-bisphosphate (PI(4,5)P₂). In contrast, the mutation of K220 and K222 that are putatively involved in 3′-phosphate binding should have a much greater effect on binding to PIP₃ than binding to PI(4,5)P₂. Our SPR analysis showed that the K220A/K222A mutation had a much larger effect on binding to PIP₃-containing vesicles, causing the mutant to lose selectivity for PIP₃ over PI(4,5)P₂. In contrast, the K172A mutation lowered the affinity of Syk-cSH2 for PIP₃- and PI(4,5)P₂-containing vesicles similarly and thus the mutant retained the selectivity of Syk-cSH2 wild type (WT) for PIP₃ over PI(4,5)P₂. Lastly, mutation of R195 in the pY-binding pocket to Ala did not affect the membrane binding properties of Syk-cSH2, confirming that the pY binding pocket is not involved in lipid binding. We also determined the affinity of Syk-cSH2 WT and mutants for a fluorescein-labeled pY-containing peptide derived from a B cell co-receptor, Igα, by fluorescence anisotropy as reported previously²¹. Results showed that the K220A/K222A mutation did not alter the pY-peptide affinity of Syk-cSH2 whereas the mutation of the pY site (i.e., R195A) lowered the peptide affinity by two orders of magnitude. Collectively, this unprecedented atomic-level consistency between computational and experimental data demonstrates the power and accuracy of our computational prediction of the lipid binding site of Syk-cSH2 and the mode of membrane-protein interaction.

Adopting the same approach combining the computational prediction and the mutational analysis, we also determined the PIP₃-binding sites for BLNK-SH2 and PLCγ2-cSH2, respectively. Results show that unlike the PH domains that have structurally similar deep cationic PIP₃-binding pockets, the SH2 domains have much more variable PIP₃-binding sites. This structural variability of the three PIP₃-binding sites also suggests that they may be specifically blocked by small molecule inhibitors with low to no cross-reactivity.

### PIP₃ Binding of Syk-cSH2 is Important for Cellular Function of Syk

A chicken B cell line, DT40, has been a boon to BCR signaling research because of availability and easiness of preparation of numerous derivatives deficient of BCR signaling proteins^{42,43}. To evaluate the importance of Syk-cSH2-PIP₃ binding in the cellular function of Syk, we transfected Syk^{-/-} DT40 cells with full-length (FL) Syk WT and various mutants and compared their cellular properties. First, we transiently transfected Syk^{-/-} DT40 cells with EGFP-Syk-WT and EGFP-Syk-K220A/K222A, respectively. It has been generally thought that upon BCR stimulation recruitment of Syk to the BCR signaling complex at the plasma membrane is driven by binding of its tandem SH2 domains to phosphorylated B cell co-receptors, Igα and Igβ³⁰. When Syk^{-/-} DT40 cells co-transfected with EGFP-Syk WT and a genetically encoded biosensor for PIP₃, mCherry-eMyoX-tPH⁴⁴, were stimulated with a plate-coated BCR antigen, IgM, which leads to phosphorylation of Igα and Igpβ³⁰, EGFP-Syk-WT and mCherry-eMyoX-tPH were spontaneously co-localized at the plasma membrane. Plasma membrane translocation of EGFP-Syk WT was greatly suppressed by either elimination of IgM or inhibition of PIP₃ synthesis by a Class I PI3K inhibitor, GDC-0941, showing that it requires both IgM stimulation and PIP₃ at the plasma membrane. These results also preclude the possibility that membrane localization of EGFP-Syk WT was non-specifically driven by protein overexpression. Most importantly, EGFP-Syk-K220A/K222A largely remained in the cytosol even with IgM stimulation and PIP₃ at the plasma membrane although the SH2 domains of this mutant have intact affinity for the phosphorylated B cell co-receptors. These results indicate that PIP₃ binding is essential for the plasma membrane recruitment of Syk upon BCR stimulation.

We also compared the BCR signaling activity of Syk^{-/-} DT40 cells stably transfected with Syk-WT and Syk-K220A/K222A, respectively by measuring the increase in the cellular Ca²⁺ concentration ([Ca²⁺]ᵢ) in response to BCR stimulation. [Ca²⁺]ᵢ is a common readout of BCR signaling activity as the activation of the Syk-BLNK-Btk-PLCγ2 signaling axis leads to an increase in [Ca²⁺]ᵢ. BCR stimulation by IgM did not raise [Ca²⁺]ᵢ in Syk^{-/-} DT40 cells whereas it sharply enhanced [Ca²⁺]ᵢ in Syk^{-/-} DT40 cells stably expressing Syk WT. Under the same conditions, a greatly attenuated [Ca²⁺]ᵢ response was observed in Syk^{-/-} DT40 cells stably expressing Syk-K220A/K222A although expression levels of Syk-WT and Syk-K220A/K222A were comparable in these cells. Given that Syk-WT and Syk-K220A/K222A have essentially identical affinity for the phosphorylated B cell co-receptors, these results underscore the importance of Syk-cSH2-PIP₃ binding in the BCR signaling function of Syk. Collectively, these results show that PIP₃ binding of Syk-cSH2 is pivotal for the BCR signaling activity of Syk and suggest that inhibition of Syk-cSH2-PIP₃ binding should have strong biological effects.

### SH2 Domain-Lipid Binding can be Specifically Inhibited by Small Molecules

Despite high variability of the PIP₃-binding sites of the three SH2 domains, some general features emerged: i.e., these sites are made of shallow and wide grooves. This finding hinted that some non-lipidic small molecule scaffolds may efficiently bind these pockets and inhibit SH2-PIP₃ binding. Using the PIP₃-binding pockets of the SH2 domains as templates, we thus performed high-throughput molecular docking of a series of candidate compounds with different scaffolds.

We also optimized a recently developed fluorescence quenching-based lipid-binding assay⁴⁷ for high-throughput screening of these molecules for their inhibitory activity against the PIP₃-SH2 interaction. Specifically, we expressed SH2 domains as EGFP-tagged proteins and directly quantified their membrane binding by monitoring the quenching of EGFP fluorescence emission intensity by a dark quencher-containing lipid, N-dimethylaminoazo-benzenesulfonyl-phosphatidylethanolamine (dabsyl-PE), incorporated in the vesicles. We then screened for compounds that inhibited binding of Syk-cSH2, BLNK-SH2, and PLCγ2-cSH2, respectively, to 1-palmitoyl-2-oleoyl-PC (POPC)/1-palmitoyl-2-oleoyl-PS (POPS)/PIP₃/dabsyl-PE (67:20:3:10) vesicles while eliminating those that inhibit their non-specific binding to anionic POPC/POPS/dabsyl-PE (70:20:10) vesicles. The quality of the screening system was evaluated using 1% dimethylsulfoxide (DMSO) and 20 μM D-myo-inositol-1,3,4,5-tetraphosphate (IP₄) as negative and positive controls, respectively. The clear separation of positive and negative control data and the average Z′-factor of 0.7 supported the robustness of our system for high-throughput screen of PIP₃-SH2 interaction inhibitors.

Multiple rounds of screening yielded lead compounds for each SH2 domain.

TABLE 1

| | 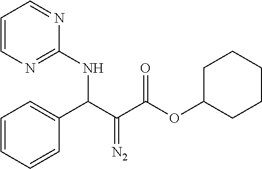 | 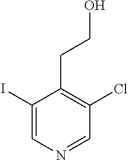 [SMILES: OCCc1c(Cl)cncc1I] | 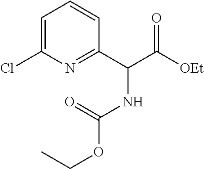 [SMILES: CCOC(=O)NC(C(=O)OCC)c1cccc(Cl)n1] |
|---|---|---|---|
| Name | VG354 | VG594 | VG370 |
| Target | Syk-cSH2 | BLNK-SH2 | PLCy2-cSH2 |

TABLE 2

| | VG354 | | VG594 | | VG370 | |
|---|---|---|---|---|---|---|
| | Iₘₐₓ (%) | IC₅₀ (nM) | Iₘₐₓ (%) | IC₅₀ (nM) | Iₘₐₓ (%) | IC₅₀ (nM) |
| Syk-cSH2 | 40 ± 5 | 350 ± 20 | <10 | ND^{a} | <10 | ND |
| BLNK-SH2 | <10 | ND | 45 ± 5 | 400 ± 30 | <20 | ND |
| PLCγ2-cSH2 | <10 | ND | <10 | ND | 50 ± 5 | 680 ± 50 |

^{a}Not determined

Unlike competitive inhibitors of enzymes, these molecules caused varying degrees of maximal inhibition at saturating concentrations depending on the mode and efficiency of their inhibitory action. It is because the membrane binding sites of soluble proteins typically comprise a pocket that specifically binds a cognate lipid and the flanking surface that makes non-specific, mostly electrostatic, contact with the membrane⁴⁸. Since the latter type of interaction also contributes to the overall membrane affinity of these proteins⁴⁸ and would not be blocked by inhibitors targeting specific lipid pockets, some of these proteins may be able to interact with the membrane to some degree even in the presence of an inhibitor targeting the lipid-binding pocket. We thus defined two parameters to assess their inhibitory efficacy: maximal inhibition (Iₘₐₓ) and the concentration required for half-maximal inhibition (IC₅₀). The lead compounds (VG354, VG594, and VG370) had IC₅₀ values around 500 nM and achieved 40-50% Iₘₐₓ and showed a high degree of specificity even before optimization. We further tested their specificity in Raji B lymphocytes. Specifically, we measured the effects of the inhibitors on the BCR signaling axis (i.e., Syk-BLNK-PLCγ2) by Western blot analysis. An inhibitor of Syk (VG354), which acts at the top of the signaling axis, suppressed phosphorylation of Syk (pY525/pY526), BLNK (pY96) and PLCγ2 (pY759). Furthermore, the BLNK-SH2 inhibitor (VG594) blocked the phosphorylation of BLNK and PLCγ2 without a significant effect on Syk phosphorylation whereas the inhibitor for PLCγ2 (VG370), which sits at the bottom of the signaling cascade, selectively suppressed phosphorylation of PLCγ2. These results verify the specificity of these SH2 domain inhibitors under physiological conditions.

For Syk-cSH2, we optimized VG354 by the structure-activity relationship analysis. Out of 39 derivatives of VG354, we found three structurally related compounds, WC35, WC36, and WC38, with greatly improved inhibitory activity (Tables 3 and 4). Structures derivatives of VG354 and their efficacy determined by the vesicle binding assay (Table 3).

TABLE 3

| | |
|---|---|
| 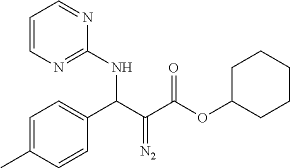 | VG-354 |
| Iₘₐₓ (%) = 40 | |
| IC₅₀ (μM) = 0.5 | |
| 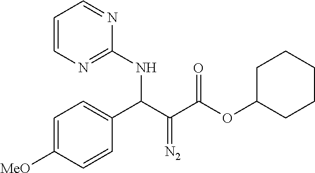 | WC-30 |
| Iₘₐₓ (%) = 10 | |
| IC₅₀ (μM) = 3.8 | |
| 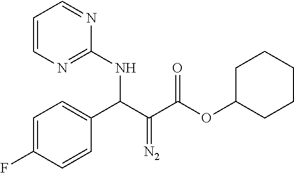 | WC-32 |
| Iₘₐₓ (%) = 10 | |
| IC₅₀ (μM) = 5 | |
| 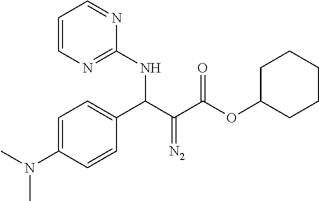 | WC-34 |
| Iₘₐₓ (%) = 10 | |
| IC₅₀ (μM) = 2.8 | |
| 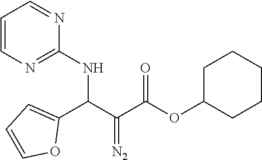 | WC-35 |
| Iₘₐₓ (%) = 50 | |
| IC₅₀ (μM) = 0.15 | |
| 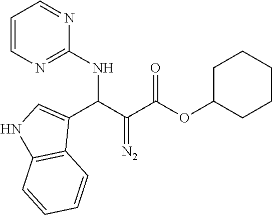 | WC-36 |
| Iₘₐₓ (%) = 60 | |
| IC₅₀ (μM) = 0.04 | |
| 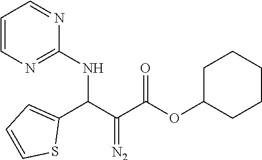 | WC-37 |
| Iₘₐₓ (%) = 15 | |
| IC₅₀ (μM) = 2.8 | |
| 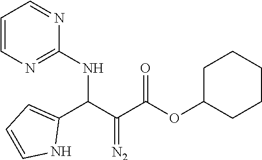 | WC-38 |
| Iₘₐₓ (%) = 70 | |
| IC₅₀ (μM) = 0.12 | |
| 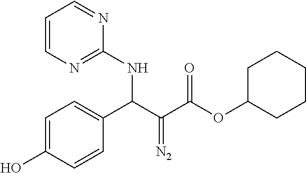 | WC = 42 |
| Iₘₐₓ (%) = 12 | |
| IC₅₀ (μM) = 4 | |
| 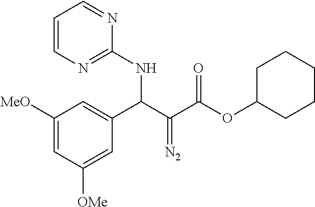 | WC-45 |
| Iₘₐₓ (%) = 12 | |
| IC₅₀ (μM) = 3.6 | |
| 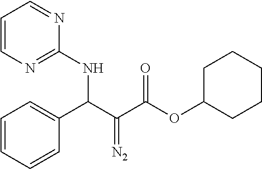 | WC-46 |
| Iₘₐₓ (%) = 10 | |
| IC₅₀ (μM) = 5 | |
| 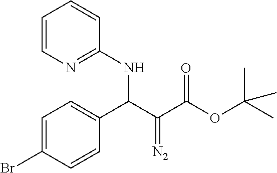 | WC-50 |
| Iₘₐₓ (%) = 10 | |
| IC₅₀ (μM) = 4.5 | |
| 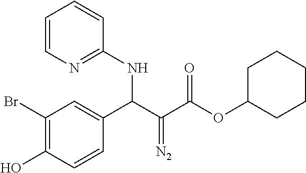 | WC-52 |
| Iₘₐₓ (%) = 12 | |
| IC₅₀ (μM) = 3.5 | |
| 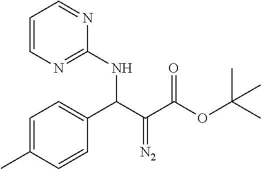 | WC-56 |
| Iₘₐₓ (%) ≤ 10 | |
| IC₅₀ (μM) = 2 | |
| 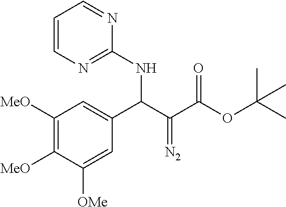 | WC-59 |
| Iₘₐₓ (%) ≤ 10 | |
| IC₅₀ (μM) = 4 | |
| 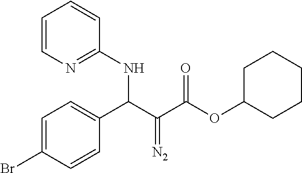 | WC-62 |
| Iₘₐₓ (%) ≤ 10 | |
| IC₅₀ (μM) = 5 | |
| 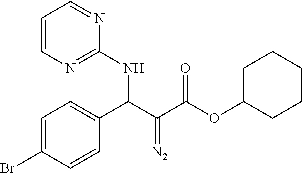 | WC-68 |
| Iₘₐₓ (%) ≤ 10 | |
| IC₅₀ (μM) = 4.5 | |
| 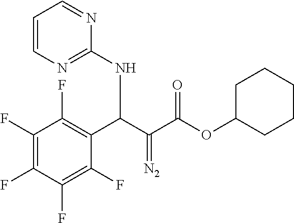 | WC-70 |
| Iₘₐₓ (%) ≤ 10 | |
| IC₅₀ (μM) = 2.5 | |
| 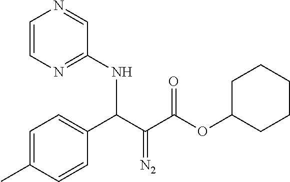 | WC-73 |
| Iₘₐₓ (%) ≤ 10 | |
| IC₅₀ (μM) = 8 | |
| 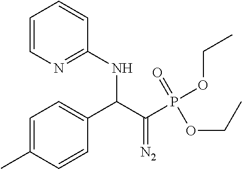 | WC-75 |
| Iₘₐₓ (%) = 12 | |
| IC₅₀ (μM) = 7 | |
| 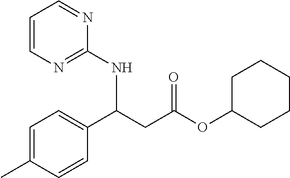 [SMILES: Cc1ccc(C(CC(=O)OC2CCCCC2)Nc2ncccn2)cc1] | WC-79 |
| Iₘₐₓ (%) ≤ 10 | |
| IC₅₀ (μM) = 10 | |
| 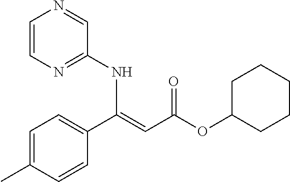 [SMILES: Cc1ccc(/C(=C/C(=O)OC2CCCCC2)Nc2cnccn2)cc1] | WC-80 |
| Iₘₐₓ (%) = > 10 | |
| IC₅₀ (μM) = 15 | |
| 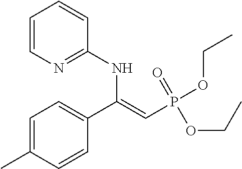 [SMILES: CCOP(=O)(/C=C(\Nc1ccccn1)c1ccc(C)cc1)OCC] | WC-85 |
| Iₘₐₓ (%) ≤ 10 | |
| IC₅₀ (μM) = 12 | |
| 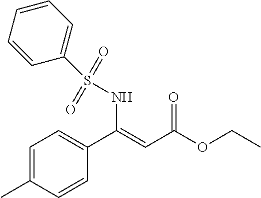 [SMILES: CCOC(=O)/C=C(\NS(=O)(=O)c1ccccc1)c1ccc(C)cc1] | WC-90 |
| Iₘₐₓ (%) ≤ 10 | |
| IC₅₀ (μM) = 20 | |
| 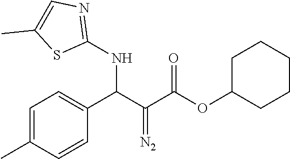 | WC-92 |
| Iₘₐₓ (%) = 15 | |
| IC₅₀ (μM) = 10 | |
| 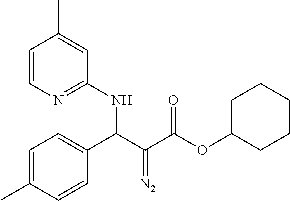 | WC-95 |
| Iₘₐₓ (%) ≤ 10 | |
| IC₅₀ (μM) = 5 | |
| 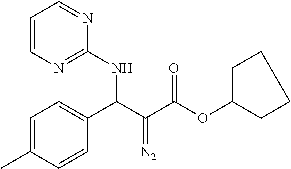 | WC-100 |
| Iₘₐₓ (%) = 18 | |
| IC₅₀ (μM) = 2 | |
| 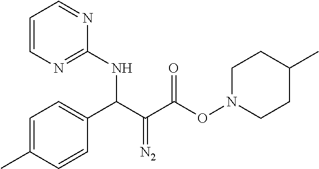 | WC-105 |
| Iₘₐₓ (%) = 15 | |
| IC₅₀ (μM) = 4 | |
| 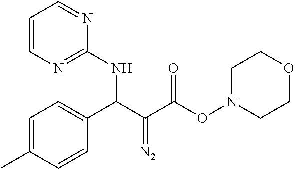 | WC-108 |
| Iₘₐₓ (%) = 20 | |
| IC₅₀ (μM) = 2 | |
| 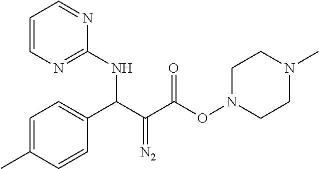 | WC-109 |
| Iₘₐₓ (%) = 25 | |
| IC₅₀ (μM) = 2 | |
| 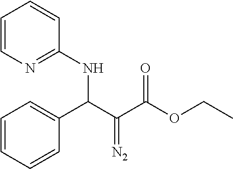 | WC-110 |
| Iₘₐₓ (%) ≤ 10 | |
| IC₅₀ (μM) = 5 | |
| 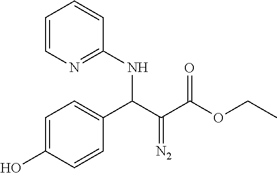 | WC-111 |
| Iₘₐₓ (%) ≤ 10 | |
| IC₅₀ (μM) = 7 | |
| 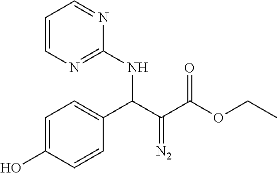 | WC-115 |
| Iₘₐₓ (%) = 20 | |
| IC₅₀ (μM) = 2 | |
| 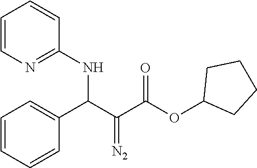 | WC-118 |
| Iₘₐₓ (%) = 15 | |
| IC₅₀ (μM) = 4 | |
| 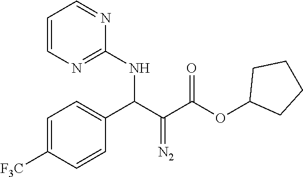 | WC-120 |
| Iₘₐₓ (%) = 20 | |
| IC₅₀ (μM) = 4 | |
| 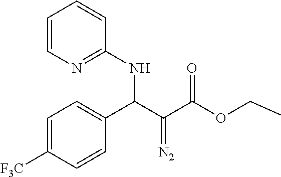 | WC-121 |
| Iₘₐₓ (%) ≤ 10 | |
| IC₅₀ (μM) = 4.8 | |
| 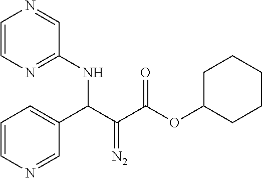 | WC-125 |
| Iₘₐₓ (%) = 20 | |
| IC₅₀ (μM) = 1.5 | |
| 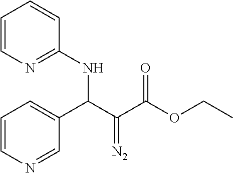 | WC-128 |
| Iₘₐₓ (%) = 18 | |
| IC₅₀ (μM) = 3.5 | |
| 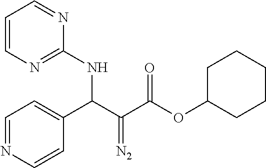 | WC-130 |
| Iₘₐₓ (%) = 25 | |
| IC₅₀ (μM) = 2 | |
| 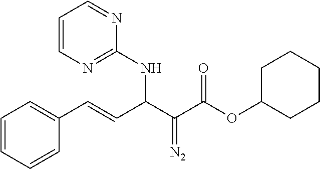 | WC-132 |
| Iₘₐₓ (%) = 15 | |
| IC₅₀ (μM) = 5 | |

TABLE 4

| | | WC35 | | WC36 | | WC38 | |
|---|---|---|---|---|---|---|---|
| Assays | Proteins | Iₘₐₓ (%) | IC₅₀ (nM) | Iₘₐₓ (%) | IC₅₀ (nM) | Iₘₐₓ (%) | IC₅₀ (nM) |
| Vesicle | Syk-cSH2 | 50 ± 5 | 150 ± 25 | 60 ± 5 | 40 ± 5 | 70 ± 5 | 120 ± 10 |
| binding^{a} | BLNK-SH2 | <10 | ND^{d} | <10 | ND | <10 | ND |
| | PLCγ2-cSH2 | <10 | ND | <10 | ND | <10 | ND |
| | ZAP70-cSH2 | <20 | ND | <10 | ND | 30 ± 5 | 340 ± 20 |
| Immuno- | pSyk^{b} | 58 ± 5 | 180 ± 50 | 67 ± 5 | 52 ± 10 | 65 ± 3 | 180 ± 30 |
| blotting | pZAP70^{c} | <10 | ND | <10 | ND | <10 | ND |

^{a}POPC/POPS/PIP₃/dabsyl-PE (67:20:3:10) vesicles (40 μM) were used for the assay.
^{b}Determined from the Western blot assay in Raji B cells.
^{c}Determined from the Western blot assay in Jurkat T cells.
^{d}Not determined

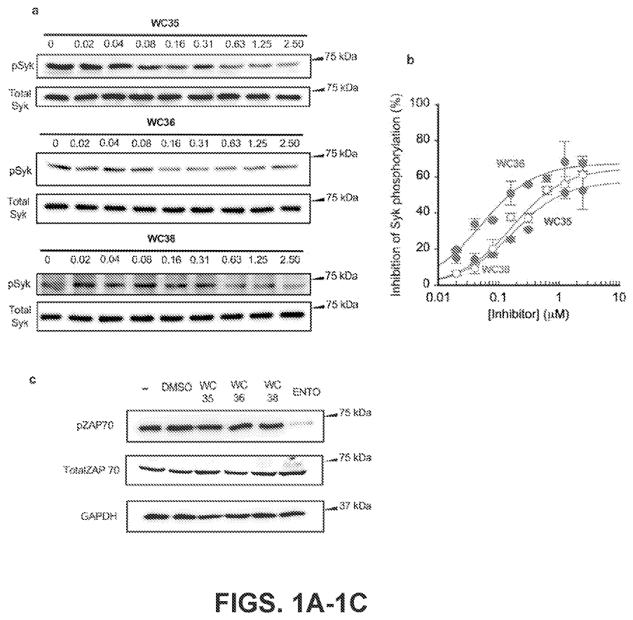

These compounds inhibited binding of neither BLNK-SH2 nor PLCγ2-cSH2 to POPC/POPS/PIP₃/dabsyl-PE vesicles, underscoring their specificity (Table 3). We then measured activity of these optimized compounds in Raji B cells. WC35, WC36, and WC38 effectively suppressed phosphorylation of all BCR signaling proteins. Their Iₘₐₓ and IC₅₀ values determined from the dose-dependent inhibition of Syk phosphorylation (FIGS. 1a and 1b and Table 4) show that WC35, WC36, and WC38 potently block BCR-mediated activation of Syk in B cells. Molecular docking calculations suggested that WC36 might tightly bind to the primary PIP₃ pocket of Syk-cSH2 in an orientation that would block the entry of PIP₃ to the pocket. In this binding mode, WC36 makes multiple hydrogen bonds with the residues in the pocket.

ZAP-70 is another Syk family kinase primarily found in T cells and is structurally and functionally similar to Syk⁴⁹. In the vesicle binding assay, WC35, WC36, and WC38 did not inhibit binding of ZAP-70-cSH2 to POPC/POPS/PIP₃/dabsyl-PE vesicles, showing their selectivity for Syk-cSH2 over ZAP-70-cSH2 (Table 3). We also treated Jurkat T cells that contain Zap70 in lieu of Syk. Immunoblotting of T cell receptor signaling proteins showed that WC35, WC36, and WC38 did not inhibit phosphorylation and activation of ZAP-70 (FIG. 1c and Table 3). In contrast, entospletinib, which is currently the most potent and specific ATP-competitive Syk inhibitor³⁵, significantly inhibited ZAP-70 phosphorylation as reported previously⁵⁰. As far as the off-target toxicity is concerned, our inhibitors thus offer an important therapeutic advantage over entospletinib. This also underscores the major advantage of targeting highly variable lipid binding sites of the SH2 domains over targeting structurally similar ATP binding sites of kinase domains.

### New Syk Inhibitors Serve as Potent and Resistance-Proof Drugs Against AML Cells

Clinical efficacy of Syk inhibitors for hematologic malignancies has been evaluated as either monotherapy or combined therapy with other kinase inhibitors or chemotherapy agents³⁵. Despite a central role Syk plays in BCR signaling, clinical benefits of Syk inhibitors in B cell malignancies have not been demonstrated to date³⁵. However, multiple lines of preclinical and clinical data support Syk as a therapeutic target in AML. AML is a cancer of the myeloid line of blood cells, characterized by the rapid proliferation of poorly differentiated myeloid cells that build up in the bone marrow and blood and inhibit normal hematopoiesis^{51,52}. AML had been typically treated with chemotherapy but new therapy targeting tyrosine kinases has been introduced in past few years^{53,54}. This therapy is based on the finding that in up to one third of AML cells FMS-like tyrosine kinase 3 receptor (FLT3) is constitutively activated through mutation, leading to proliferation and survival of AML cells^{54,55}. Syk inhibition has recently emerged as a promising targeted approach for AML patients with the hyperactivated FLT3⁵⁶ based on the report that hyperactivated FLT3 in AML cells still depends on Syk for driving myeloid neoplasia in mice⁵⁷. It was also reported that high levels of Syk phosphorylation in AML bone marrow specimens is a poor prognostic marker⁵⁸. Two orally bioavailable Syk inhibitors, entospletinib and TAK-659, have entered clinical trials for patients with AML, with both studies demonstrating early evidence of response, including a modest number of complete responses with single-agent treatment^{56,59}. As with targeted therapy for other kinases²⁷, however, Syk-targeted therapy for AML is associated with the rapid emergence of resistance³⁷. Among downstream signaling pathways of Syk in AML cells, including the PI3K-AKT-mTOR, JAK-STAT, and RAS-RAF-MEK-ERK pathways, the main resistance mechanism to Syk kinase inhibition primarily involves alternate activation of the RAS-RAF-MEK-ERK signaling pathway³⁷. Thus, RAS-mutated AML cells show de novo resistance to Syk kinase inhibitors whereas AML cells with WT RAS quickly develop acquired resistance through mutation(s) of RAS or other proteins regulating RAS activity, such as PTPN11 and CBL³⁷.

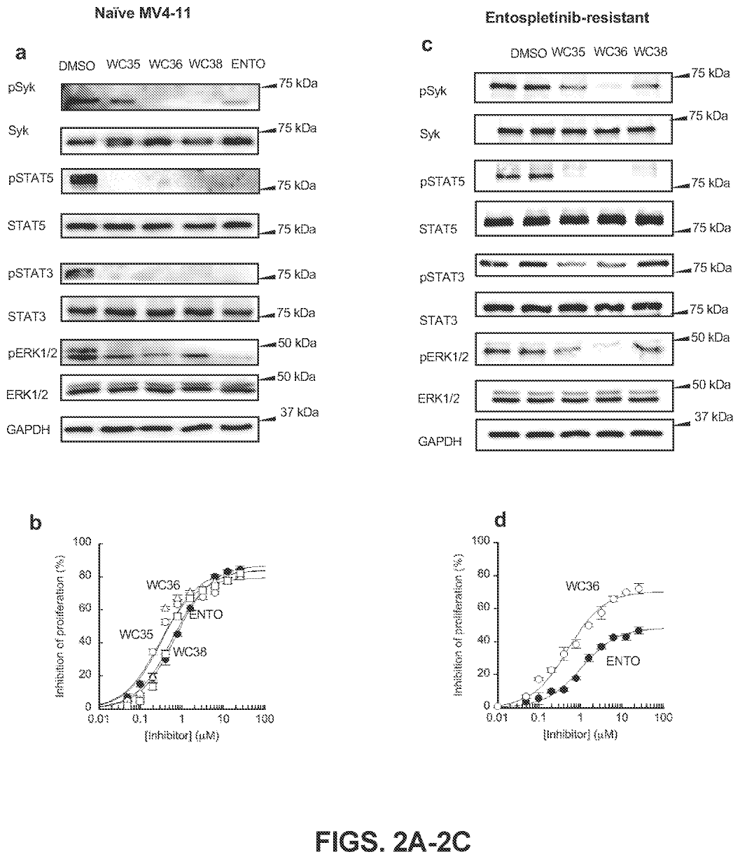

We first assessed the efficacy of our Syk inhibitors against AML using MV4-11 AML cells that harbor WT RAS and oncogenic FLT3 mutation⁶⁰. Although the mutated FLT3 in MV4-11 cells is constitutively active (i.e., active without a FLT3 ligand), its full signaling activity still requires Syk activation⁵⁷. The mechanism of Syk activation in AML cells is not fully understood but it has been reported that stimulation of Fcγ receptor I (FcγRI) or integrin receptor activates Syk in several AML cells lines and induces cell survival and proliferation⁵⁵. We thus activated Syk signaling in MV4-11 with an antibody specific for FcγRI (i.e., IgG2), which strongly induced phosphorylation of Syk, STAT3/5, and ERK1/2. WC36 and WC38 suppressed phosphorylation of Syk and STAT3/5 as potently as entospletinib but were modestly less active than entospletinib for ERK1/2 inhibition (FIG. 2a). WC35 was less active than entospletinib for both Syk and ERK1/2 inhibition. When the effects of the inhibitors on the cell proliferation activity were measured by the XTT assay, WC35 and WC36 were modestly more active than WC38 and entospletinib (FIG. 2b and Table 4). Overall, against MV4-11 cells these compounds showed potency comparable to entospletinib.

We then put selection pressure on MV4-11 cells by growing them under chronic treatment of inhibitors with gradually increasing concentrations from 0.5 to 5 μM over 10 weeks according to a reported protocol³⁷. Entospletinib-resistant MV4-11 cells started to emerge after 4 weeks under these conditions and those surviving cells were able to proliferate in the presence of 5 μM entospletinib. It was reported that ERK1/2 and STAT3/5 in entospletinib-resistant MV4-11 cells were activated by alternate signaling pathways bypassing Syk kinase activity³⁷. Consistent with this notion, immunoblotting analysis showed that 5 μM entospletinib could not inhibit phosphorylation of ERK1/2 and STAT3/5 in resistant MV4-11 cells while still potently inhibiting Syk phosphorylation. In contrast, 5 μM WC36 potently suppressed the phosphorylation and activation of Syk, ERK1/2, and STAT3/5 in entospletinib-resistant MV4-11 cells (FIG. 2c). WC35 and WC38 were significantly less active than WC36 in these cells. The XTT assay also showed that entospletinib could not effectively suppress the proliferation of these cells (Iₘₐₓ≤50%) even at high concentrations (i.e., ≥20 μM) whereas WC36 inhibited their proliferation almost as potently as naïve MV4-11 cells (i.e., IC₅₀=0.50±0.01 μM; Iₘₐₓ≃71±2%) (FIG. 2d). We then checked if MV4-11 cells could acquire resistance to WC36 under the same conditions in which they developed resistance to entospletinib. Interestingly, we could not detect any surviving MV4-11 cells under these conditions (i.e., increasing WC36 from 0.5 to 5 μM over 10 weeks), indicating that MV4-11 cells could not develop resistance to WC36 while readily developing resistance to entospletinib under the same conditions. This was not due to non-specific cytotoxicity of WC36 because 0.5-5 μM of WC36 showed a minimal effect on cell viability of Raji B cells. These results demonstrate that WC36 potently inhibits the signaling pathways leading to acquired resistance to entospletinib. They also suggest that binding of Syk to PIP₃ in the plasma membrane is still crucial for the Syk kinase-independent signaling pathways leading to entospletinib resistance.

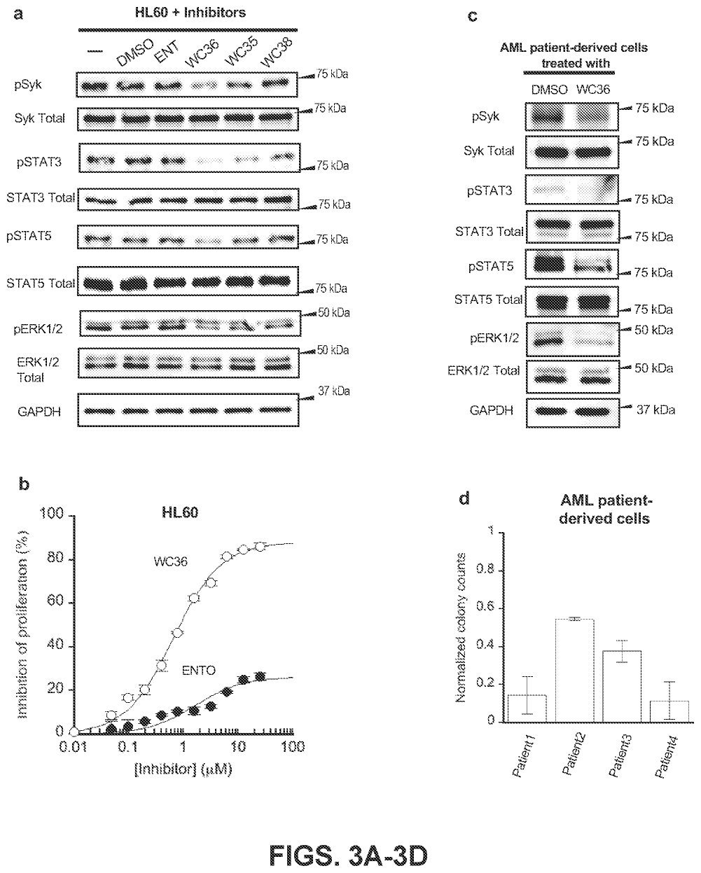

High efficacy of WC36 against entospletinib-resistant MV4-11 cells also suggested that it might be active against other RAS-mutated AML cells. We thus measured the efficacy of our inhibitors and entospletinib against RAS-mutated HL60 cells. As reported previously³⁷, entospletinib could not suppress phosphorylation of ERK1/2 and STAT3/5 in HL60 cells (FIGS. 3a and 3b). However, WC36 was potent against phosphorylation of ERK1/2 and STAT3 under the same conditions (FIGS. 3a and 3b). WC35 and WC38 were less active then WC36. Also, WC36 potently suppressed the proliferation of HL60 cells while entospletinib was ineffective. These results demonstrate that WC36 is refractory to not only acquired resistance but also de novo resistance. This novel property should confer on WC36 high efficacy against diverse AML cells.

To test the clinical benefit of these desirable inhibitory properties of WC36, we tested its efficacy against primary myeloid cells collected from AML patients with different genetic backgrounds. AML cells from 4 patients refractory or relapsed following standard AML therapies all showed significant inhibition of Syk activation in response to WC36 treatment (FIGS. 3c and 3d) and this correlated with statistically significant suppression of colony forming activity. Collectively, these results demonstrate that our new inhibitor targeting Syk-cSH2-PIP₃ binding not only is specific and potent in suppressing diverse AML cells, including relapsed/refractory patient samples, but also offers major advantages over conventional Syk inhibitors targeting the kinase domain in that it abrogates both de novo and acquired drug resistance by AML cells.

### Non-Catalytic Scaffolding Function of Syk is Essential for the Acquired Resistance Mechanism to Syk Kinase Inhibitors.

To understand why WC36 is refractory to the acquired drug resistance response by AML cells while entospletinib is highly susceptible, we performed two lines of experiments. First, we genetically modulated Syk in entospletinib-resistant MV4-11 cells. shRNA-mediated suppression of Syk abrogated phosphorylation of ERK1/2 and STAT3/5, confirming that Syk is still required for their activation in these cells. We then reintroduced to Syk-deficient entospletinib-resistant MV4-11 cells WT and mutant Syk carrying silent mutations to avoid gene suppression by the shRNA for endogenous Syk. Reintroduction of WT or a kinase-inactive mutant of Syk (Y525A/Y526A)⁶¹ into Syk-deficient MV4-11 cells rescued phosphorylation of ERK1/2 and STAT3/5 whereas that of K220A/K222A, which has compromised PIP₃ binding activity, did not under the same conditions. These results show that Syk-cSH2-PIP₃ binding is directly involved in and essential for non-catalytic activation of ERK1/2 and STAT3/5 by Syk in entospletinib-resistant MV4-11 cells, presumably through the scaffolding function of Syk.

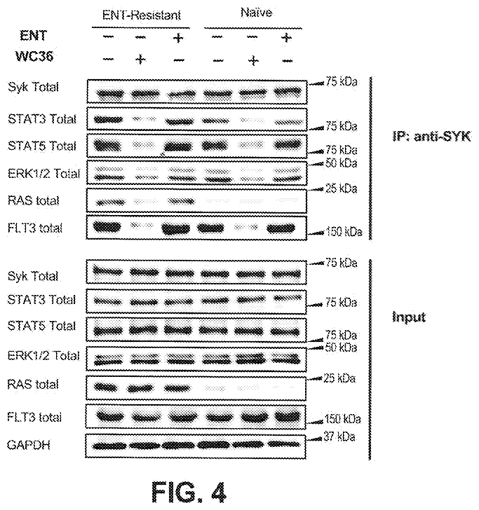

We then performed immunoprecipitation of Syk in entospletinib-resistant MV4-11 cells to test if Syk serves as a scaffolding protein to activate ERK1/2 and STAT3/5 in these cells. Immunoblotting analysis showed that STAT3/5 and the proteins in the MAP kinase axis, including Ras and ERK1/2, were co-precipitated with Syk. FLT3 was also co-precipitated with Syk. These co-immunoprecipitation signals were not due to non-specific backgrounds as demonstrated by a control experiment with beads alone. Most importantly, 5 μM WC36 greatly suppressed co-precipitation whereas 5 μM entospletinib did not (FIG. 4). These data strongly support the notion that Syk serves as a scaffold protein that interacts with FLT3, ERK1/2 and STAT3/5 directly or via an adaptor protein(s) in entospletinib-resistant MV4-11 cells.

We also performed the same sequence of experiments in naïve MV4-11 cells. As was the case with entospletinib-resistant MV4-11 cells, FLT3, ERK1/2 and STAT3/5 were co-precipitated with Syk in naïve MV4-11 cells (FIG. 4). The only difference was that RAS was not found in the immuno-precipitated complex and that the RAS expression level was much lower in naïve MV4-11 cells than in entospletinib-resistant MV4-11 cells (see input in FIG. 4). This suggests that Syk coordinates interaction of amplified RAS with FLT3 and other proteins to activate MAP kinase signaling in a Syk kinase-independent manner in entospletinib-resistant MV4-11 cells. Again, 5 μM WC36 greatly suppressed the scaffolding function of Syk whereas 5 μM entospletinib had little to no effect in naïve MV4-11 cells (FIG. 4). Collectively, these results support the notion that WC36 is refractory to acquired resistance because it effectively blocks the non-catalytic scaffolding function of Syk that plays a pivotal role in alternate activation of ERK1/2 and STAT3/5 and resistance to ATP-competitive Syk inhibitors.

WC36 Specifically Interacts with and Inhibits Syk in AML Cells.

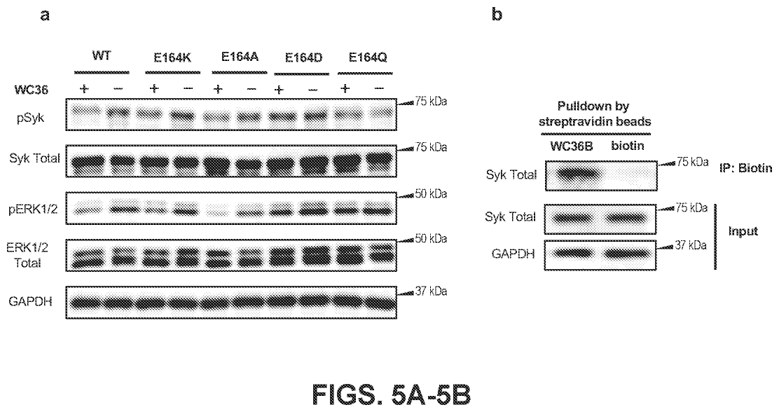

To prove that WC directly and specifically interacts with and inhibits Syk in AML cells, we performed two independent experiments. First, we generated Syk-cSH2 mutants that might have the same membrane binding activity as WT but might not be inhibited by WC36. These mutants were designed on the basis of our molecular dynamics simulation and molecular docking suggesting that WC36 binds deeper to the primary lipid pocket than the PIP₃ headgroup and that some residues, most notably E164, in the pocket are exclusively involved in WC36 binding. Four mutants of E164 were expressed as EGFP-fusion proteins and their membrane binding activity was evaluated by the fluorescence quenching assay using POPC/POPS/PIP₃/dabsyl-PE vesicles. Among these mutants, E164D had the same affinity as WT but unlike WT was not inhibited by up to 5 μM WC36. E164Q was also refractory to WC36 inhibition although it had slightly lower membrane affinity than WT. When the full-length E164D and E164Q, respectively, were introduced into Syk-deficient MV4-11 cells, WC36 did not inhibit phosphorylation of these mutants and ERK1/2 (FIG. 5a). WC36 potently suppressed phosphorylation of Syk WT and ERK1/2 when Syk WT was added back to the Syk-deficient AML cells.

We also prepared a biotinylated derivative of WC36 (WC36B) and incubated MV4-11 cells with 5 μM WC36B for 24 h, enriched the WC36B-bound proteins by pulldown with streptavidin beads, and analyzed the captured proteins by immunoblotting.

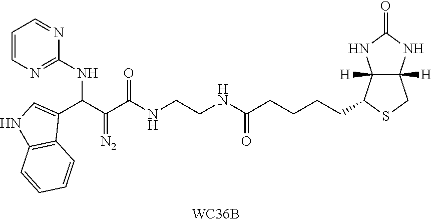

Clearly, the Syk band was visible in the blot (FIG. 5b), demonstrating that WC36B targets Syk in MV4-11 cells. To prove that WC36 targets not the ATP-binding site of Syk but its primary PIP₃ binding cavity, we performed the WC36B-streptavidin pull down assay in the presence of WC36, entospletinib, and ATP, respectively. As shown in FIG. 5b, only WC36 was able to compete with WC36B for Syk binding, displacing Syk from the immobilized WC36B. This shows that WC36 binds specifically to the lipid-binding pocket, but not to the ATP-binding pocket, of Syk. This competition result also precludes the possibility that WC36 binds to Syk covalently due to the presence of α-diazo ester functionality.

To verify the specificity of WC36B-Syk binding, we then performed mass spectrometry-based proteomics analysis of WC36B-binding cellular proteins captured by streptavidin beads using biotin as a negative control. Comparative analysis of WC36B- and biotin-bound proteins showed that Syk is the single major protein that is captured by the WC36 pharmacophore. Although several other proteins were also identified in the WC36B-captured mixture, they were found in much lower levels than Syk. Altogether, these results verify the cellular specificity of WC36-Syk interaction.

### Discussion

Despite their potentials, developing lipid-protein interaction inhibitors as drugs remain elusive. Due to the well-documented implication of PIP₃ in human diseases, early efforts to develop lipid-protein interaction inhibitors focused on identifying lipid-like molecules that can block binding of PH domains to PIP₃¹³. However, these attempts have met with limited success for many practical reasons. In particular, structural similarity within the canonical lipid binding domain families, such as PH domains^{7,14}, and the difficulty in generating libraries of diverse lipid-like molecules¹³ hampered the progress. The present study takes a completely different approach of targeting the lipid binding sites of SH2 domains, which are a prototypal protein interaction domain¹⁸⁻²⁰ that also interacts with membrane lipids²¹⁻²³. The strength and novelty of our approach derive from the fact that lipids control not only the enzymatic activity of SH2 domain-containing kinases (and other proteins) but also the SH2 domain-coordinated protein-protein interaction network²¹⁻²³. Thus, inhibiting lipid binding would block the source of diverse functions of these proteins, including non-catalytic scaffolding functions, which is difficult to achieve with active site-directed reagents. As elaborated below, the fundamental and essential role of SH2 domain-lipid interaction in diverse signaling cascades make it difficult for cells to develop resistance to SH2 domain-lipid binding inhibitors because any escape mutation or alternate mechanism would lead to cell dysfunction and death. The successful development of a specific and potent Syk inhibitor serves as a proof of concept for our new strategy.

The SH2 domains of key B cell signaling proteins, Syk, BLNK, and PLCγ2, tightly bind PIP₃ in the plasma membrane, which is crucial for the B cell signaling activity. Although it has been recognized that cytosolic proteins undergo significant conformational changes when they bind to membrane lipids^{7,15,38}, the lack of high-resolution structures of membrane-bound cytosolic proteins has made it extremely difficult to rationally design molecules that efficaciously block lipid binding of these proteins in the membrane environment. Our computational approach using the HMMM model rapidly samples a large number of membrane-bound conformations of a protein in a realistic membrane environment, thereby providing us with the atomic-level insight into how specifically and differently these SH2 domains bind the membrane and interact with PIP₃ therein. The primary PIP₃-binding sites of the membrane-bound SH2 domains identified by molecular dynamics simulations are not fully formed in their crystal or solution structures, underscoring the difficulty in predicting them from the reported structures. Importantly, unlike PIP₃-binding PH domains that have a common structure with a deep cationic pocket^{7,14}, the SH2 domains have variable yet generally shallow and wide grooves that interact with PIP₃ in distinctively different modes, suggesting that they may be specifically targeted by non-lipidic small molecules.

The valuable new mechanistic insight from our computational analysis also enabled us to generate a new small molecule library for lipid-protein interaction inhibitors and rationally design and optimize them through the structure-activity relationship analysis and molecular docking. The shallow lipid binding grooves of SH2 domains can be partially occupied by a group of molecular scaffolds whose derivatives are readily accessible by modular synthesis. Despite its relatively small size, the utility of our new library is validated by the successful identification of selective inhibitors for all three tested SH2 domains from the library. Each of these inhibitors selectively blocks PIP₃ binding of the target SH2 domain and also selectively inhibits the cellular activity of its host protein. Furthermore, optimization of the lead compound for Syk led to the most potent and specific inhibitor for Syk, WC36, with exceptional inhibitory activity.

Syk plays important roles in many hematologic cells, including B cells, macrophages and mast cells³⁰. Dysregulation of Syk in different hematologic cells leads to various diseases, including cancer and autoimmune diseases and thus Syk has been an attractive target for drug development³⁵. Most of the currently available Syk inhibitors target its ATP-binding site in the kinase domain³⁵. WC36 is modestly more potent than or as active as one of the best available ATP-competitive Syk inhibitors, entospletinib, in Raji B cells and AML myeloblast cells. Furthermore, WC36 shows much higher specificity than entospletinib as demonstrated by its specificity for Syk over ZAP-70, a closely related T cell counterpart of Syk⁴⁹. Most importantly, WC36 is superior to entospletinib in terms of invulnerability to both de novo and acquired drug resistance. First, WC36 is effective against RAS-mutated HL-60 AML cells that shows de novo resistance to entospletinib. Second, MV4-11 AML cells do not develop acquired resistance to WC36 under the same conditions they readily develop resistance to entospletinib³⁷. Furthermore, WC36 can potently inhibit those AML cells that have already developed resistance to entospletinib. Entospletinib-resistant MV4-11 AML cells have acquired an ability to activate ERK1/2 and STAT3/5 and proliferate independently of the Syk kinase activity, thereby making Syk kinase inhibitors ineffective. It was reported that hyperactivation of ERK1/2 derived from mutations in the upstream RAS signaling pathway, which bypasses Syk³⁷. However, our results show that entospletinib-resistant MV4-11 AML cells still depend on the presence of Syk for their survival as their proliferative signaling activity is abrogated by shRNA-based Syk knockdown. Importantly, the fact that this Syk knockdown can be fully rescued by Syk WT and a kinase-inactive mutant, but not by a PIP₃ binding-deficient Syk mutant, shows that the PIP₃-mediated non-catalytic activity of Syk is crucial for a new signaling pathway leading to activation of ERK1/2 and STAT3/5 and resistance to entospletinib. In the resting state, Syk is autoinhibited by intramolecular tethering by interdomain regions which occludes the kinase domain and prohibits the scaffolding function by the SH2 domains (FIG. 5d). Our results show that PIP₃-dependent membrane binding of Syk is necessary and sufficient to induce conformational changes of Syk to activate its scaffolding function even when its active site is blocked (FIG. 5d). They also suggest that kinase-independent scaffolding activity of Syk coordinates MAP kinase and STAT signaling networks that lay the foundation for the acquired resistance mechanism to ATP-competitive Syk inhibitors (FIG. 5d). Most importantly, they demonstrate that blocking SH2-lipid interaction is a highly efficient approach to suppressing all Syk-related cellular deregulation.

Extensive studies on the drug resistance mechanism, which have mainly focused on transmembrane receptor tyrosine kinases, have revealed diverse and complex mechanisms by which cancer cells acquire resistance to drugs targeting these kinases⁴⁻⁶. While much is now known about the resistance mechanisms for receptor tyrosine kinase drugs, which include mutation of target kinases and activation of alternate signaling pathways⁴⁻⁶, less is known about those for non-receptor kinases. One of the best studied drug resistance mechanisms for non-receptor kinases involves Src, which is also a SH2 domain-containing tyrosine kinase involved in cancer and other diseases⁶², Recent studies showed that conventional ATP-competitive Src inhibitors could actually promote drug resistance by locking the kinase in an active conformation, which in turn facilitates non-catalytic, SH2 domain-mediated scaffolding function of Src,^{28,29}. As described above, the acquired resistance to ATP-competitive Syk inhibitors, including entospletinib, appears to be developed by the same mechanism (FIG. 5d). Furthermore, it has been reported that many non-receptor kinases have kinase-independent scaffolding function⁶³⁻⁶⁵. This in turn suggests that other kinase inhibitors may facilitate drug resistance by the same or a similar mechanism, underscoring an urgent need for alternate approaches to the kinase inhibitor discovery. Selective inhibition of the inactive conformation of kinases has been suggested as an alternate strategy and produced some clinically active inhibitors, as exemplified by imatinib that targets the inactive conformation of BCR-Abl kinase⁶⁶. However, they are in general difficult to develop because of low availability of structural data on inactive protein conformations and difficulties associated with high-throughput screening for this type of inhibitors⁶⁷⁻⁶⁹. By comparison, our strategy of targeting lipid binding of SH2 domains of kinases is a generally applicable and easily accessible approach to discovery of specific, potent, and resistance-defying kinase inhibitors. It is well known that many SH2 domain-containing proteins serve as scaffolding or adaptor proteins¹⁸⁻²⁰. Our recent studies have also shown that lipids coordinate and regulate scaffolding function of many signaling proteins constituting signaling complexes at the plasma membrane^{70,71}. Taken together, we speculate that it is common for cells to take advantage of the lipid-mediated kinase-independent scaffolding function of SH2 domain-containing kinases to develop resistance to their active site-directed inhibitors. In this regard, we expect that our new approach of targeting drug resistance at the source will yield potent, specific, and resistance-proof inhibitors for many other cytosolic kinases harboring SH2 domains.

In conclusion, our work demonstrates that targeting lipid binding of SH2 domain-containing kinases, many of which are implicated in human diseases, is a promising new approach to developing novel small molecule drugs. Our Syk inhibitor shows exceptional inhibitory activity because of its ability to block both catalytic and non-catalytic cellular Syk activities. As such, it represents the first successful development of a non-lipidic lipid-protein interaction inhibitor for kinases. Given that lipids also control functions of diverse cell signaling proteins containing the SH2 domain²¹⁻²³, including non-enzymic, scaffolding proteins, such as BLNK, for which little to no option for inhibitor development is available, our strategy is expected to generate potent and specific inhibitors for many proteins, including those hitherto considered undruggable. With the increase in our knowledge on the mechanisms of the dynamic lipid-protein interaction in the membrane environment and the expansion of small molecule libraries covering more chemical space, our approach can also be applied to a broader range of lipid-binding signaling proteins beyond SH2 domain-containing proteins.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Dang, C. V., Reddy, E. P., Shokat, K. M. & Soucek, L. Drugging the ‘undruggable’ cancer targets. Nat Rev Cancer 17, 502-508 (2017).
2. Lazo, J. S. & Sharlow, E. R. Drugging Undruggable Molecular Cancer Targets. Annu Rev Pharmacol Toxicol 56, 23-40 (2016).
3. Rudolph, J., Settleman, J. & Malek, S. Emerging Trends in Cancer Drug Discovery—From Drugging the “Undruggable” to Overcoming Resistance. Cancer Discov 11, 815-821 (2021).
4. Vasan, N., Baselga, J. & Hyman, D. M. A view on drug resistance in cancer. Nature 575, 299-309 (2019).
5. Sun, C. & Bernards, R. Feedback and redundancy in receptor tyrosine kinase signaling: relevance to cancer therapies. Trends Biochem Sci 39, 465-74 (2014).
6. Holohan, C., Van Schaeybroeck, S., Longley, D. B. & Johnston, P. G. Cancer drug resistance: an evolving paradigm. Nat Rev Cancer 13, 714-26 (2013).
7. Cho, W. & Stahelin, R. V. Membrane-protein interactions in cell signaling and membrane trafficking. Annu Rev Biophys Biomol Struct 34, 119-51 (2005).
8. Saliba, A. E., Vonkova, I. & Gavin, A. C. The systematic analysis of protein-lipid interactions comes of age. Nat Rev Mol Cell Biol 16, 753-61 (2015).
9. Di Paolo, G. & De Camilli, P. Phosphoinositides in cell regulation and membrane dynamics. Nature 443, 651-7 (2006).
10. Kihara, Y. Introduction: Druggable Lipid Signaling Pathways. Adv Exp Med Biol 1274, 1-4 (2020).
11. Nawrotek, A. et al. PH-domain-binding inhibitors of nucleotide exchange factor BRAG2 disrupt Arf GTPase signaling. Nat Chem Biol 15, 358-366 (2019).
12. Burgett, A. W. et al. Natural products reveal cancer cell dependence on oxysterol-binding proteins. Nat Chem Biol 7, 639-47 (2011).
13. Meuillet, E. J. Novel inhibitors of AKT: assessment of a different approach targeting the pleckstrin homology domain. Curr Med Chem 18, 2727-42 (2011).
14. Lemmon, M. A. Membrane recognition by phospholipid-binding domains. Nat Rev Mol Cell Biol 9, 99-111 (2008).
15. Muller, M. P. et al. Characterization of Lipid-Protein Interactions and Lipid-Mediated Modulation of Membrane Protein Function through Molecular Simulation. Chem Rev 119, 6086-6161 (2019).
16. Attwood, M. M., Fabbro, D., Sokolov, A. V., Knapp, S. & Schioth, H. B. Trends in kinase drug discovery: targets, indications and inhibitor design. Nat Rev Drug Discov 20, 839-861 (2021).
17. Pawson, T. & Nash, P. Assembly of cell regulatory systems through protein interaction domains. Science 300, 445-52 (2003).
18. Pawson, T. Specificity in signal transduction: from phosphotyrosine-SH2 domain interactions to complex cellular systems. Cell 116, 191-203 (2004).
19. Lim, W. A. & Pawson, T. Phosphotyrosine signaling: evolving a new cellular communication system. Cell 142, 661-7 (2010).
20. Liu, B. A. & Nash, P. D. Evolution of SH2 domains and phosphotyrosine signalling networks. Philos Trans R Soc Lond B Biol Sci 367, 2556-73 (2012).
21. Park, M. J. et al. SH2 Domains Serve as Lipid-Binding Modules for pTyr-Signaling Proteins. Mol Cell 62, 7-20 (2016).
22. Sheng, R. et al. Lipids Regulate Lck Protein Activity through Their Interactions with the Lck Src Homology 2 Domain. J Biol Chem 291, 17639-50 (2016).
23. Kim, E. et al. Cellular phosphatase activity of C1-Ten/Tensin2 is controlled by Phosphatidylinositol-3,4,5-triphosphate binding through the C1-Ten/Tensin2 SH2 domain. Cell Signal 51, 130-138 (2018).
24. Kraskouskaya, D., Duodu, E., Arpin, C. C. & Gunning, P. T. Progress towards the development of SH2 domain inhibitors. Chem Soc Rev 42, 3337-70 (2013).
25. Levitzki, A. Tyrosine kinase inhibitors: views of selectivity, sensitivity, and clinical performance. Annu Rev Pharmacol Toxicol 53, 161-85 (2013).
26. Roskoski, R., Jr. Classification of small molecule protein kinase inhibitors based upon the structures of their drug-enzyme complexes. Pharmacol Res 103, 26-48 (2016).
27. Roskoski, R., Jr. Properties of FDA-approved small molecule protein kinase inhibitors. Pharmacol Res 144, 19-50 (2019).
28. Higuchi, M. et al. Paradoxical activation of c-Src as a drug-resistant mechanism. Cell Rep 34, 108876 (2021).
29. Temps, C. et al. A Conformation Selective Mode of Inhibiting SRC Improves Drug Efficacy and Tolerability. Cancer Res 81, 5438-5450 (2021).
30. Mocsai, A., Ruland, J. & Tybulewicz, V. L. The SYK tyrosine kinase: a crucial player in diverse biological functions. Nat Rev Immunol 10, 387-402 (2010).
31. Herzog, S., Reth, M. & Jumaa, H. Regulation of B-cell proliferation and differentiation by pre-B-cell receptor signalling. Nat Rev Immunol 9, 195-205 (2009).
32. Pierce, S. K. & Liu, W. The tipping points in the initiation of B cell signalling: how small changes make big differences. Nat Rev Immunol 10, 767-77 (2010).
33. Rickert, R. C. New insights into pre-BCR and BCR signalling with relevance to B cell malignancies. Nat Rev Immunol 13, 578-91 (2013).
34. Burger, J. A. & Wiestner, A. Targeting B cell receptor signalling in cancer: preclinical and clinical advances. Nat Rev Cancer 18, 148-167 (2018).
35. Liu, D. & Mamorska-Dyga, A. Syk inhibitors in clinical development for hematological malignancies. J Hematol Oncol 10, 145 (2017).
36. Sharman, J. & Di Paolo, J. Targeting B-cell receptor signaling kinases in chronic lymphocytic leukemia: the promise of entospletinib. Ther Adv Hematol 7, 157-70 (2016).
37. Cremer, A. et al. Resistance Mechanisms to SYK Inhibition in Acute Myeloid Leukemia. Cancer Discov 10, 214-231 (2020).
38. Wen, P. C. et al. Microscopic view of lipids and their diverse biological functions. Curr Opin Struct Biol 51, 177-186 (2018).
39. Ohkubo, Y. Z., Pogorelov, T. V., Arcario, M. J., Christensen, G. A. & Tajkhorshid, E. Accelerating membrane insertion of peripheral proteins with a novel membrane mimetic model. Biophys J 102, 2130-9 (2012).
40. Soubias, O. et al. Membrane surface recognition by the ASAP1 PH domain and consequences for interactions with the small GTPase Arf1. Sci Adv 6(2020).
41. Narula, S. S. et al. Solution structure of the C-terminal SH2 domain of the human tyrosine kinase Syk complexed with a phosphotyrosine pentapeptide. Structure 3, 1061-73 (1995).
42. Winding, P. & Berchtold, M. W. The chicken B cell line DT40: a novel tool for gene disruption experiments. J Immunol Methods 249, 1-16 (2001).
43. Yasuda, T. & Yamamoto, T. Analysis of B-cell signaling using DT40 B-cell line. Methods Mol Biol 271, 261-70 (2004).
44. Liu, S. L. et al. Quantitative Lipid Imaging Reveals a New Signaling Function of Phosphatidylinositol-3,4-Bisphosphate: Isoform- and Site-Specific Activation of Akt. Mol Cell 71, 1092-1104 e5 (2018).
45. Chernyak, D., Chernyak, N. & Gevorgyan, V. Efficient and General Synthesis of 3-Aminoindolines and 3-Aminoindoles via Copper-Catalyzed Three Component Coupling Reaction. Adv Synth Catal 352, 961-966 (2010).
46. Chernyak, N. & Gevorgyan, V. General and efficient copper-catalyzed three-component coupling reaction towards imidazoheterocycles: one-pot synthesis of alpidem and zolpidem. Angew Chem Int Ed Engl 49, 2743-6 (2010).
47. Kim, H., Afsari, H. S. & Cho, W. High-throughput fluorescence assay for membrane-protein interaction. J Lipid Res 54, 3531-8 (2013).
48. Cho, W. Building signaling complexes at the membrane. Sci STKE 2006, pe7 (2006).
49. Wang, H. et al. ZAP-70: an essential kinase in T-cell signaling. Cold Spring Harb Perspect Biol 2, a002279 (2010).
50. Colado, A. et al. The kinase inhibitors R406 and GS-9973 impair T cell functions and macrophage-mediated anti-tumor activity of rituximab in chronic lymphocytic leukemia patients. Cancer Immunol Immunother 66, 461-473 (2017).
51. Döhner, H., Weisdorf, D. J. & Bloomfield, C. D. Acute Myeloid Leukemia. N Engl J Med 373, 1136-52 (2015).
52. Yang, X. & Wang, J. Precision therapy for acute myeloid leukemia. J Hematol Oncol 11, 3 (2018).
53. Cioccio, J. & Claxton, D. Therapy of acute myeloid leukemia: therapeutic targeting of tyrosine kinases. Expert Opin Investig Drugs 28, 337-349 (2019).
54. Fernandez, S. et al. Targeting Tyrosine Kinases in Acute Myeloid Leukemia: Why, Who and How? Int J Mol Sci 20 (2019).
55. Oellerich, T. et al. beta2 integrin-derived signals induce cell survival and proliferation of AML blasts by activating a Syk/STAT signaling axis. Blood 121, 3889-99, S1-66 (2013).
56. Bartaula-Brevik, S., Lindstad Brattas, M. K., Tvedt, T. H. A., Reikvam, H. & Bruserud, O. Splenic tyrosine kinase (SYK) inhibitors and their possible use in acute myeloid leukemia. Expert Opin Investig Drugs 27, 377-387 (2018).
57. Puissant, A. et al. SYK is a critical regulator of FLT3 in acute myeloid leukemia. Cancer Cell 25, 226-42 (2014).
58. Boros, K. et al. Increased SYK activity is associated with unfavorable outcome among patients with acute myeloid leukemia. Oncotarget 6, 25575-87 (2015).
59. Page, E. C. et al. Interim Results from the IMPACT Study: Evidence for Prostate-specific Antigen Screening in BRCA2 Mutation Carriers. Eur Urol 76, 831-842 (2019).
60. Lange, B. et al. Growth factor requirements of childhood acute leukemia: establishment of GM-CSF-dependent cell lines. Blood 70, 192-9 (1987).
61. Law, C. L., Chandran, K. A., Sidorenko, S. P. & Clark, E. A. Phospholipase C-gamma1 interacts with conserved phosphotyrosyl residues in the linker region of Syk and is a substrate for Syk. Mol Cell Biol 16, 1305-15 (1996).
62. Yeatman, T. J. A renaissance for SRC. Nat Rev Cancer 4, 470-80 (2004).
63. Mohan, M. L. & Naga Prasad, S. V. Scaffolding Function of PI3Kgamma Emerges from Enzyme's Shadow. J Mol Biol 429, 763-772 (2017).
64. Martens, S., Hofmans, S., Declercq, W., Augustyns, K. & Vandenabeele, P. Inhibitors Targeting RIPK1/RIPK3: Old and New Drugs. Trends Pharmacol Sci 41, 209-224 (2020).
65. Mousson, A. et al. Targeting Focal Adhesion Kinase Using Inhibitors of Protein-Protein Interactions. Cancers (Basel) 10 (2018).
66. Schindler, T. et al. Structural mechanism for STI-571 inhibition of abelson tyrosine kinase. Science 289, 1938-42 (2000).
67. Huse, M. & Kuriyan, J. The conformational plasticity of protein kinases. Cell 109, 275-82 (2002).
68. Liu, Y. & Gray, N. S. Rational design of inhibitors that bind to inactive kinase conformations. Nat Chem Biol 2, 358-64 (2006).
69. Noble, M. E., Endicott, J. A. & Johnson, L. N. Protein kinase inhibitors: insights into drug design from structure. Science 303, 1800-5 (2004).
70. Sheng, R. et al. Cholesterol modulates cell signaling and protein networking by specifically interacting with PDZ domain-containing scaffold proteins. Nat Commun 3, 1249 (2012).
71. Sheng, R. et al. Cholesterol selectively activates canonical Wnt signalling over non-canonical Wnt signalling. Nat Commun 5, 4393 (2014).
72. Gulevich, A. V., Helan, V., Wink, D. J. & Gevorgyan, V. Pyridine group assisted addition of diazo-compounds to imines in the 3-CC reaction of 2-aminopyridines, aldehydes, and diazo-compounds. Org Lett 15, 956-9 (2013).
73. Stahelin, R. V. & Cho, W. Differential roles of ionic, aliphatic, and aromatic residues in membrane-protein interactions: a surface plasmon resonance study on phospholipases A2. Biochemistry 40, 4672-8 (2001).
74. Zhang, J. H., Chung, T. D. & Oldenburg, K. R. A Simple Statistical Parameter for Use in Evaluation and Validation of High Throughput Screening Assays. J Biomol Screen 4, 67-73 (1999).
75. Khan, I. et al. FOXM1 contributes to treatment failure in acute myeloid leukemia. JCI Insight 3 (2018).
76. Humphrey, W., Dalke, A. & Schulten, K. VMD: Visual molecular dynamics. Journal of Molecular Graphics & Modelling 14, 33-38 (1996).
77. Qi, Y. F. et al. CHARMM-GUI HMMM Builder for Membrane Simulations with the Highly Mobile Membrane-Mimetic Model. Biophysical Journal 109, 2012-2022 (2015).
78. Phillips, J. C. et al. Scalable molecular dynamics with NAMD. Journal of Computational Chemistry 26, 1781-1802 (2005).
79. Best, R. B. et al. Optimization of the Additive CHARMM All-Atom Protein Force Field Targeting Improved Sampling of the Backbone phi, psi and Side-Chain chi(1) and chi(2) Dihedral Angles. Journal of Chemical Theory and Computation 8, 3257-3273 (2012).
80. Klauda, J. B. et al. Update of the CHARMM All-Atom Additive Force Field for Lipids: Validation on Six Lipid Types. Journal of Physical Chemistry B 114, 7830-7843 (2010).
81. Essmann, U. et al. A Smooth Particle Mesh Ewald Method. Journal of Chemical Physics 103, 8577-8593 (1995).
82. Martyna, G. J., Tobias, D. J. & Klein, M. L. Constant-Pressure Molecular-Dynamics Algorithms. Journal of Chemical Physics 101, 4177-4189 (1994).
83. Wells, D. B., Abramkina, V. & Aksimentiev, A. Exploring transmembrane transport through alpha-hemolysin with grid-steered molecular dynamics. J Chem Phys 127, 125101 (2007).
84. Phillips, J. C. et al. Scalable molecular dynamics with NAMD. J Comput Chem 26, 1781-802 (2005).
85. Roos, K. et al. OPLS3e: Extending Force Field Coverage for Drug-Like Small Molecules. J Chem Theory Comput 15, 1863-1874 (2019).
86. Sastry, G. M., Adzhigirey, M., Day, T., Annabhimoju, R. & Sherman, W. Protein and ligand preparation: parameters, protocols, and influence on virtual screening enrichments. J Comput Aided Mol Des 27, 221-34 (2013).
87. Halgren, T. A. Identifying and characterizing binding sites and assessing druggability. J Chem Inf Model 49, 377-89 (2009).
88. Friesner, R. A. et al. Extra precision glide: docking and scoring incorporating a model of hydrophobic enclosure for protein-ligand complexes. J Med Chem 49, 6177-96 (2006).
89. Zhang, J. et al. Identifying mutation hotspots reveals pathogenetic mechanisms of KCNQ2 epileptic encephalopathy. Sci Rep 10, 4756 (2020).

1. A compound of formula I, or a pharmaceutically acceptable salt thereof 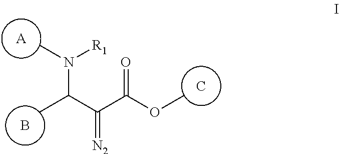
wherein 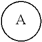
is 5 or 6 membered substituted or unsubstituted heteroaryl; 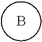
is 5 to 10 membered substituted or unsubstituted heteroaryl or substituted or unsubstituted phenyl; 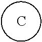
is cycloalkyl; and
R₁ is H or C₁-C₄ alkyl.

2. The compound of claim 1, wherein 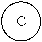
is cyclohexyl.

3. The compound of claim 1, wherein 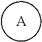
is a 6-membered substituted or unsubstituted heteroaryl.

4. The compound of claim 1, wherein

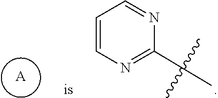

5. The compound of claim 1, wherein R¹ is H.

6. The compound of claim 1, wherein 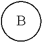
is a 5 or 9-membered substituted or unsubstituted heteroaryl.

7. The compound in any one of claims 1-5 of claim 1, wherein

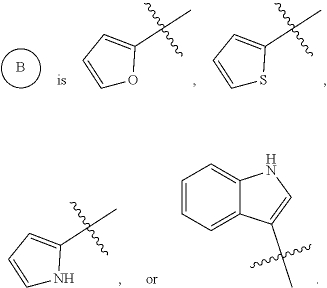

8. The compound of claim 1, wherein 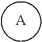
is a 6-membered substituted or unsubstituted heteroaryl, 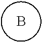
is a 5 membered substituted or unsubstituted heteroaryl or a 9 membered fused heteroaryl, 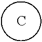
is cyclohexyl, and
R¹ is hydrogen.

9. The compound of claim 1, wherein the compound is formula II

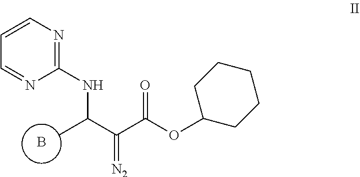

10. The compound of claim 9, wherein 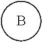
is a 5 membered substituted or unsubstituted heteroaryl.

11. The compound of claim 10, wherein the 5 membered heteroaryl is a substituted or unsubstituted furan, a substituted or unsubstituted pyrrole, or a substituted or unsubstituted thiophene.

12. The compound of claim 10, wherein the 5 membered heteroaryl is an unsubstituted furan or an unsubstituted pyrrole.

13. The compound of claim 9, wherein 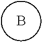
is a 9 membered fused substituted or unsubstituted heteroaryl selected from the group consisting of a substituted or unsubstituted indole, a substituted or unsubstituted isoindole, a substituted or unsubstituted indolizine, a substituted or unsubstituted purine, or a substituted and unsubstituted indole.

14. (canceled)

15. (canceled)

16. The compound of claim 1, wherein the compound is

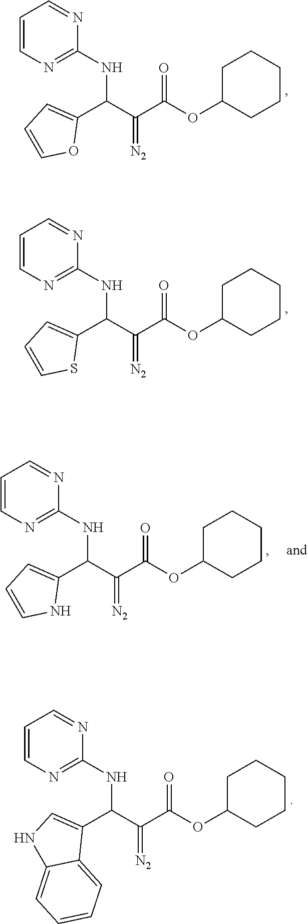

17. A pharmaceutical composition comprising a compound of claim 1 and a pharmaceutically acceptable carrier.

18. A method of treating a subject suffering from a disease or disorder for which inhibiting spleen tyrosine kinase (Syk) would provide a benefit comprising administering to the subject an effective amount of the compound of claim 1, or a pharmaceutically acceptable salt thereof.

19. The method of claim 18 wherein the disease or disorder is selected from cancer, diabetes, and immune disorders.

20. The method of claim 18, wherein the disease or disorder is selected from chronic lymphoid leukemia (CLL) or acute myeloid leukemia (AML).

21. A method of inhibiting spleen tyrosine kinase (Syk) in a cell comprising contacting the cell with an effective amount of claim 1, or a pharmaceutically acceptable salt thereof.

22. A method of treating chronic lymphoid leukemia (CLL) or acute myeloid leukemia (AML) in a subject comprising administering to the subject an effective amount of the compound of claim 1, or a pharmaceutically acceptable salt thereof.
